(19) United States
(12) Patent Application Publication
(10) Pub. No.: US 2007/0128719 A1
(43) Pub. Date: Jun. 7, 2007

(54) ISOLATION AND EXPANSION OF ANIMAL CELLS IN CELL CULTURES

(75) Inventors: Scheffer Tseng, Pinecrest, FL (US); Hua He, Miami, FL (US); Wei Li, Shenzhen (CN); Ying-Ting Chen, Miami, FL (US); Yasutaka Hayashida, Miami, FL (US)

Correspondence Address: WILSON SONSINI GOODRICH & ROSATI, 650 PAGE MILL ROAD, PALO ALTO, CA, 94304-1050, US

(73) Assignee: TissueTech, Inc., Miami, FL (US)

(21) Appl. No.: 11/617,630

(22) Filed: Dec. 28, 2006

Related U.S. Application Data
(63) Continuation-in-part of application No. 11/476,376, filed on Jun. 28, 2006.
(63) Continuation-in-part of application No. 10/833,502, filed on Apr. 28, 2004.
(60) Provisional application No. 60/695,051, filed on Jun. 29, 2005.
(60) Provisional application No. 60/695,576, filed on Jun. 30, 2005.
(60) Provisional application No. 60/703,188, filed on Jul. 28, 2005.
(60) Provisional application No. 60/473,007, filed on May 22, 2003.
(60) Provisional application No. 60/801,491, filed on May 17, 2006.

(51) Int. Cl. C12N 5/06; C12N 5/08

(52) U.S. Cl. 435/325; 435/366

(57) ABSTRACT

Described are methods for isolating/purifying and expanding animal stem cells and stem-cell-like cells. Isolation methods include conditions comprising preferentially digesting non-stem cells and non-stem-cell-like cells in a population and preferentially adhering stem cells and stem-cell-like cells in a population. Expansion methods include culturing such cells under conditions comprising modulation of TGF-β signaling, inhibition of cell signaling mediated by p38 MAP kinase using small molecular weight inhibitors, expansion of the cells on human amniotic epithelial cells as feeder layers, control of cell seeding density, control of levels of Ca²⁺ in the culture media, rapid adhesion on a substrate or by a combination of such conditions. More particularly, what is disclosed relates to methods and systems for expanding animal cells in ex vivo cell cultures, while preventing cellular differentiation, and selectively enriching stem cells. The embodiments also disclose a culture system for ex vivo expansion of limbal epithelial cells or mesenchymal cells, as well as surgical grafts made there from.

### CROSS-REFERENCE TO RELATED APPLICATION

The present application is a continuation-in-part of U.S. patent application Ser. No. 11/476,376, filed on Jun. 28, 2006, which claims the benefit of U.S. provisional patent application Nos. 60/695,051 filed Jun. 29, 2005; 60/695,576, filed Jun. 30, 2005; and 60/703,188, filed Jul. 28, 2005; the present application is also a continuation-in-part of U.S. patent application Ser. No. 10/833,502 filed on Apr. 28, 2004, which claims the benefit of U.S. provisional patent application 60/473,007, filed May 22, 2003; and the present application claims the benefit of U.S. provisional patent application 60/801,491, filed May 17, 2006.

### STATEMENT AS TO FEDERALLY SPONSORED RESEARCH

Certain aspects of this disclosure were made with United States government support under grant number RO1 EY06819 awarded by the National Institute of Health. The United States government may have certain rights in the invention.

### FIELD OF THE INVENTION

The embodiments described herein relate generally to the fields of developmental biology, cell culture, and tissue culture.

### BACKGROUND

In tissue engineering, cells are isolated or otherwise obtained from an animal source and then expanded (i.e., grown so as to produce additional cells) to produce a desired tissue. Such tissues can then be utilized for therapeutic uses. For example, cellular or cell-based therapy is the replacement of unhealthy, damaged, or diseased cells or tissues with new cells or tissues. Blood transfusions and bone marrow transplantation are prime examples of the successful application of cell-based therapeutics, but recent advances in cellular and molecular biology have expanded the potential applications of this approach to a wide variety of clinical disorders. The realization of these applications, however, depends on obtaining or culturing the cell type of interest in sufficient numbers for transplantation into the damaged or diseased tissue or organ. Theoretically, cells of interest can be explanted from an animal or human subject and introduced into a primary cell culture system for expansion. In practice, however, defining and refining the conditions that allow primary cell expansion without phenotypic changes (e.g., differentiation from an epithelial cell phenotype to a fibroblastic phenotype) has required a prodigious effort. Current methods for cell culture expansion employ the use of murine fibroblast feeder layers. One potential biohazard of using murine fibroblast feeder layers is the transmission of murine diseases, and as a result, further manipulation or use of such cells is restricted.

### SUMMARY OF THE INVENTION

Described herein are methods for tissue engineering stem cells and stem-cell-like cells, including methods for purifying/isolating stem cells and stem-cell-like cells, and methods for expanding stem cells and stem-cell-like cells. Also described herein are methods for using isolating/purified and/or expanded stem cells and stem-cell-like cells in therapeutic, cell culture, and tissue culture applications.

In one aspect described herein are methods for preferentially purifying stem cells and/or stem-cell-like cells from a population of cells (including, by way of example only, a sheet of limbal epithelial cells). Such isolation/purification methods can be combined with any of the other tissue engineering methods described herein.

In one embodiment, the method comprises differential dispase digestion. In one embodiment, the differential dispase digestion uses an enzymatic solution such as Dispase II (also known as Dispase 2) solution. As, such purified cells can be expanded using the aforementioned method (or indeed any method for expanding described herein). In a further aspect, the Dispase II solution comprises SHEM, a polyhydroxy alcohol, a sugar, or any combination thereof. Further, a culture solution containing polyhydroxy alcohol can include sorbitol, mannitol, or galactitol. In a further or alternative embodiment, the temperature for enzymatic (e.g., dispase) digestion is (a) about ambient temperature, (b) above about ambient temperature, (c) about 20° C.; (d) about 22° C.; (e) about 25° C.; (f) about 27° C.; (g) about 30° C.; (h) about 32° C.; (i) about 35° C.; (j) about 37° C.; or (k) about 39° C. In a further or alternative embodiment, the duration of enzymatic (e.g., dispase) digestion is shorter for higher temperatures and longer for lower temperatures. In further embodiments, the differential dispase digestion cells are expanded while preventing differentiation using any of the methods described herein.

In yet a further or alternative embodiment, the stem cells or stem-cell-like cells are purified by methods comprising rapid adhesion. In one embodiment are methods for purifying stem cells or stem-cell-like cells from a population of cells comprising the steps of contacting a population of cells comprising stem cells and stem-cell-like cells with a surface comprising collagen; incubating the population of cells; and removing non-adherent cells. In certain embodiments, the incubating step is less than I hour and the collagen is collagen I. In further embodiments, the “rapid-adherent cells” are expanded while preventing differentiation using any of the methods described herein.

In one embodiment, the population of cells is selected from a sheet of cells, a tissue sample, or a modified tissue sample. In further or alternative embodiment, the surface is a plastic surface. In further or alternative embodiments, the collagen is collagen I. The incubating step can be for a relatively quick period of time, including less than about 1 hour, less than about 30 minutes, less than about 20 minutes, less than about 15 minutes, less than about 10 minutes and even less than about 5 minutes. The non-adherent cells can be removed using the methods described herein or by standard techniques for removing non-adherent cells from a surface while not killing the adherent cells. The rapid adherent cells are enriched in stem cells or stem-cell-like cells and can be further expanded using the methods described herein or by other methods known in the art.

The non-adherent cells can be further used as described herein, and in particular, the non-adherent cells acquired from the aforementioned rapid adhesion methods can have additional slow adherent cells that exhibit relatively reduced proliferative potential (i.e., relative to rapid adherent cells). Such slow-adherent cells can be purified from the non-adherent cells by providing a prolonged incubation time on a collagen coated surface, followed by removal of the non-adherent cells and expanded.

In yet a further embodiment are methods comprising differential dispase digestion and rapid adhesion and/or slow-adhesion. In further embodiments, the purified cells are expanded while preventing differentiation using any of the methods described herein.

In another aspect are methods for expanding cell cultures or tissue cultures and increasing proliferation of stem cells or stem-cell-like cells therein while at the same time decreasing the differentiation tendencies of these same cells through external cues.

One method for preventing differentiation while expanding animal cells comprises modulating TGF-β signaling in the animal cells. Cells or tissues, isolated from an animal subject, are placed in an ex vivo culture system in which the conditions are such as to downregulate TGF-β signaling in the cells. Also described are methods for expanding limbal epithelial progenitor cells (isolated or in tissues) while preventing cellular differentiation comprising modulating p38 MAP kinase activity (and in one embodiment, such modulation is by means of an inhibitor of p38 MAP kinase). Also described are in vitro methods for using human amniotic epithelial cells (HAEC) to expand limbal epithelial progenitor cells (isolated or in tissues) while inhibiting differentiation of such cells.

In one embodiment for expanding animal cells (isolated or in tissues) while preventing undesired cellular differentiation, TGF-β signaling is modulated using various agents. Types of cells that can be expanded using TGF-β modulation include, but are not limited to, differentiated cells, keratocytes, stem-cell-like cells, and stem cells (including limbal epithelial progenitor cells, umbilical cord epithelial cells, and amniotic membrane epithelial cells).

In a further embodiment of this method, the culture system for expanding the animal cells comprises a culture vessel, a matrix and a medium. In one embodiment, the medium is essentially free of amniotic membrane (or “AM”; the terms AM and amniotic membrane are used interchangeably herein) and non-human mesenchymal feeder cells and the conditions are such as to downregulate TGF-β signaling in the cells. Such down regulation allows cells to proliferate without undergoing a change in their differentiation state. In a further or alternative embodiment, the media of the culture comprises low amounts of Ca²⁺ and is essentially serum-free. TGF-β can be downregulated by utilizing Ca²⁺ concentrations less than about 0.1 mM Ca²⁺ (e.g., using KSFM). In an alternative embodiment, TGF-β can be downregulated when serum is in the media and Ca²⁺ concentrations are greater than about 1.0 mM (as high as about 1.8 mM in media such as DMEM and SHEM) during or before the cultured cells are in contact with an agent that downregulates TGF-β signaling.

In a further aspect, a means for preventing differentiation of an expanded cell line occurs by contacting the cells in the cell line with agents that suppress TGF-β or by removing an agent/condition that upregulates TGF-β signaling. Agents that downregulate TGF-β signaling to promote cell expansion without differentiation include, but are not limited to: agents that specifically bind TGF-β; agents that antagonize a receptor for TGF-β; AM derivatives; purified components of AM; isolated AM; AM stromal matrices; processed AM; AM extracts (AME); components derived from AM such as hyaluronic acid (HA), HA-inter-α-trypsin-inhibitor heavy chain (HA-ITI), lumican, TSG-6, Pentraxin (PTX3) and Thrombospondin; anti-TGF-β antibodies; and inhibitors of components in the TFG-β signaling pathway such as serine/threonine kinase inhibitors and agents that prevent Smad protein translocation.

In another aspect, methods are described which feature an ex vivo cell culture system comprising a vessel. Such a vessel includes animal cells (isolated or in tissues) whose differentiation state is controllable by modulating TGF-β signaling. Further, the animal cells have been expanded by culturing the cells in a medium free of amniotic membrane (AM) under conditions which downregulate TGF-β signaling in the cells to allow the cells to proliferate without changes to their phenotype.

In another embodiment, ex vivo methods are described for preferentially expanding limbal epithelial progenitor cells (isolated or in tissues) in a cell culture. Limbal epithelial progenitor cells are expanded by combining such cells with transient amplifying cells (TACs) in an ex vivo culture system. The culture system described further comprises a culture vessel, a matrix, and a medium. The mixture of cells is then seeded in the culture system at a sufficiently low cell density (e.g., between about 10 and 1,000 cells/cm²) for a time period exceeding the lifespan of the TACs (e.g., greater than about 3 weeks). Such a time frame prevents TACs from having a negative paracrine effect on limbal epithelial progenitor cells.

In another aspect, methods are described for facilitating the outgrowth of epithelial cell sheet formation from limbal explants (isolated cells or tissues), while inhibiting abnormal epidermal cell differentiation. These methods comprise culturing the cells on a matrix in a culture medium with an effective amount of a p38 MAP kinase inhibitor. In a further embodiment, the p38 MAP kinase inhibitor is a pyridinylimidazole compound or a 2,4,5-triarylimidazole. In a further embodiment, the p38 MAP kinase inhibitor is chosen from at least one of: SB 203580, L-167307, SKF 86002, SB 220025, SB 235699, VX-745, RWJ 68354, SB 202190, SB 239063, and 2-(4-Chloro-phenyl)-4-(4-fluoro-phenyl)-5-pyridin-4-yl-1,2-dihyrdro-pyrazol-3-one.

In another aspect, ex vivo method are described for expanding limbal epithelial stem cells (isolated or in tissues) obtained from the limbus from a donor eye. The limbus is combined with an enzymatic solution in order to separate the limbal epithelial cells from the stroma of the limbus. The limbal epithelial sheet is then put in contact with an amniotic membrane (including isolated AM or AM stromal matrices). The limbal epithelial sheet-amniotic membrane composite is then cultured for a period of time and under conditions sufficient to enable the epithelial stem cells to expand.

In another embodiment, an ex vivo method for expanding mesenchymal cells (isolated or in tissues) is disclosed. The method disclosed includes contacting an amniotic membrane with at least one type of mesechymal cell in order to form a composite of the mesenchymal cell and the amniotic membrane. The composite can then be cultured in a medium for a period of time and under conditions sufficient to enable the mesenchymal cells to expand. The conditions allow for the expansion of the mesenchymal cells, yet the conditions enable the cells to maintain their current differentiated state without further unwanted differentiation. In a further aspect, keratocytes are the cells to be expanded using this method.

Also described herein are surgical grafts comprising expanded cells or tissues, prepared or cultured as described herein, including expanded limbal epithelial cells or tissues and mesenchymal cells or tissues.

In another aspect, what is described are methods for in vitro expansion of animal cells comprising seeding such animal cells on human amniotic epithelial cell feeder layers obtained from the amnion layer of human placenta. The human amniotic epithelial cell feeder layers can be grown by culturing the cells in a serum-containing media. In one embodiment such a media is supplemented hormonal epithelial media (SHEM). Further, this media can contain a calcium concentration of about 1.05 mM.

In a further aspect, the human amniotic epithelial cell feeder layers can be made from MMC-treated human amniotic epithelial cells. The cells types that can be grown on these amniotic epithelial cell feeder layer include by way of example only stem cells, stem-cell-like cells, limbal epithelial progenitor cells, umbilical cord epithelial cells, and amniotic membrane epithelial cells. Limbal epithelial progenitor cells can be obtained from the corneoscleral tissue.

As used herein, the term “cell” refers to an isolated cell, to a cell in an isolated tissue, to a cell being cultured as described herein, or to a cell in a tissue being cultured as described herein.

As used herein, the phrase “stem cell” means a cell that retains the ability to divide and differentiate into other cell types. A stem cell can be totipotent, pluripotent, or multipotent; the term “stem cell” includes progenitor cells.

The term “stem-cell-like cells” includes cells that are equivalent to progenitor cells and differentiated cells that can undergo further differentiation. One, non-limiting example of a stem-cell-like cell are keratocytes that can further differentiate into other cell types such as fibroblasts.

As used herein, the phrase “amniotic membrane derivatives” or “AM derivatives” refers to agents, materials, and compositions derived from amniotic membrane, including the jelly portion of amniotic tissue. The phrase includes the following: extracts of amniotic membrane and purified components of amniotic membranes; isolated AM; AM stromal matrices; processed AM; AM extracts (AME); and components derived from AM such as hyaluronic acid (HA), HA-inter-α-trypsin-inhibitor heavy chain (HA-ITI), lumican, TSG-6, Pentraxin (PTX3) and Thrombospondin.

By the phrase “differentiated cell” is meant a cell that is more differentiated than the stem cell from which it originated. An example of a differentiated cell is a keratocyte, which expresses cellular markers not expressed by the stem cells from which the keratocytes originated.

When referring to a cell culture system “essentially free of” a substance (e.g., intact AM or feeder cells) is meant that that substance is not present in a sufficient amount to exert a detectable effect on the cells in the culture system (e.g., to cause or prevent a phenotypic change in the cells).

As used herein, an “antibody” is an intact immunoglobulin or an antigen-binding fragment or derivative thereof

When referring to a cell, tissue, protein or other biological molecule or sample, “purified” means separated from components that naturally accompany such molecules, cells, tissues or samples. Typically, a molecule is purified when it is at least 30% (e.g., 40%, 50%, 60%, 70%, 80%, 90%, and 100%), by weight, free from the proteins or other naturally-occurring organic molecules with which it is naturally associated. Purity can be measured by any appropriate method, e.g., column chromatography, polyacrylamide gel electrophoresis, or HPLC analysis. An isolated cell or tissue is a cell or tissue that has been removed from an animal using methods described herein or known in the art.

Unless otherwise defined, all technical and legal terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All publications mentioned herein are incorporated by reference in their entirety. In the case of conflict, the present specification, including definitions will control. In addition, the particular embodiments discussed below are illustrative only and not intended to be limiting.

### BRIEF DESCRIPTION OF THE DRAWINGS

The foregoing and other objects, features, and advantages of the disclosure will be apparent from the following non-limiting illustrative embodiments of the disclosure. The drawings are not necessarily to scale, emphasis instead being placed upon illustrating the principles of the invention.

FIG. 1 is a non-limiting example of a microphotograph at 400× magnification of an immunohistochemistry stained explant cultured on dAM in a SHEM medium with addition of p38 inhibitor SB203580 at 2 weeks and at 4 weeks.

FIG. 2 is a non-limiting example of a microphotograph at 100× magnification (A, B, C, and D), and 400× magnification (E and F) of immunohistochemistry stained explants cultured in SHEM medium in the airlift manner without p38 inhibitor (FIGS. 2A, 2C, and 2E) and with p38 inhibitor (FIGS. 2B, 2D, and 2F).

FIG. 3 is a non-limiting example of a microphotograph of cultured peripheral corneal epithelial cells. FIGS. 3A-3C are microphotographs of stained cultured peripheral corneal epithelial cells showing normal differentiation in the submerge manner. FIG. 3D is a microphotograph of stained cultured peripheral corneal epithelial cells using the airlift method with a p38 MAPK inhibitor.

FIG. 4 is a non-limiting example of a microphotograph of cultured peripheral corneal epithelial cells. FIGS. 4A-4C are microphotographs of stained cultured peripheral corneal epithelial cells showing normal differentiation in the submerge manner (FIG. 4A) and in the airlift manner with p38 (FIG. 4C); and some abnormal differentiation when cultured in the airlift manner without p38 (FIG. 4B).

FIG. 5 is a non-limiting example of a bar graph demonstrating the growth capacity of human amniotic epithelial cells in all experimental conditions. HAEC cells growth was most proliferative in SHEM medium containing FBS and Ca²⁺.

FIG. 6 is a non-limiting example of a bar graph demonstrating the percentage of CK12-expressing cells in limbal clones cultured on HAEC or 3T3 feeder layers (*p<0.05, n=3). CK12 is a marker for corneal epithelium; absence of this marker for cells seeded on HAEC feeder layers demonstrates that there was less differentiation in these cells as compared to cells seeded on murine feeder layers.

FIG. 7 is a non-limiting example of a series of bar graphs demonstrating the percentages of p63-, Musashi-1-, or ABCG2-positive cells in limbal clones on HAEC or 3T3 feeder layers (*p<0.05, n=4). p63-, Musashi-1, and ABCG2 are all stem cell markers. The higher percentage of stem cell markers on cells seeded on HAECs demonstrates that cells seeded on HAEC feeder layers inhibits differentiation of stem cells to a greater extent than the same cells seeded on murine feeder layers.

### DETAILED DESCRIPTION OF THE INVENTION

Described herein are methods for tissue engineering stem cells and/or stem-cell-like cells comprising (a) methods for isolating/purifying stem cells and/or stem-cell-like cells, (b) methods for expanding stem cells and/or stem-cell-like cells, and (c) combinations of such methods. Thus, described are methods for preferentially purifying a population of stem cells or stem-cell-like cells from a sample (e.g., a tissue sample). Such methods include differential dispase digestion, rapid adhesion methods, and combinations thereof. Also described herein are systems and methods for expanding primary animal cells (isolated or in tissues) without undesired differentiation of their phenotype (i.e., preventing undesired differentiation). The systems and methods described herein involve manipulating cell signaling to control the differentiation of cells (including stem cells and stem-cell-like cells) from one phenotype to another. In particular, methods disclosed herein include at least one of the following steps: downregulation of TGF-β signaling to prevent the differentiation of a cell during expansion; methods for controlling differentiation by inhibiting the p38 MAP kinase pathway; expansion of epithelial cells or mesenchymal cells using AM derivatives; controlling expansion of epithelial stem cells through the use of human amniotic epithelial cell (HAEC) feeder layers; adjusting Ca²⁺ levels in the media; control of cell seeding density; or a combination of the foregoing.

By way of example, the following embodiments illustrate adaptation of these compositions and methods, but are in no way limiting.

### Biological Methods

Methods involving conventional biological techniques are described herein. Such techniques are generally known in the art and are described in detail in methodology treatises such as Molecular Cloning: A Laboratory Manual, 3^{rd} ed., vol. 1-3, ed. Sambrook et al., Cold Spring Harbor Laboratory Press, Cold Spring Harbor, N.Y., 2001; and Current Protocols in Molecular Biology, ed. Ausubel et al., Greene Publishing and Wiley-Interscience, New York, 2003 (with periodic updates). Various conventional techniques for culturing animal cells are described in Culture of Animal Cells: A Manual of Basic Technique, 4^{th} ed., R. Ian Freshney, Wiley-Liss, Hoboken, N.J., 2000, and Animal Cell Culture Techniques (Springer Lab Manual), M. Clynos, Springer-Verlag, New York, N.Y., 1998. Methods involving protein analysis and purification are also known in the art and are described in Protein Analysis and Purification: Benchtop Techniques, 2^{nd} ed., Ian M. Rosenberg, Birkhauser, New York, N.Y., 2004.

### Ex Vivo Methods of Expanding Animal Cells without Differentiation by Downregulation or Inhibition of TGF-β Cell Signaling Pathways

One aspect described herein includes methods of expanding animal cells ex vivo whose differentiation state is controllable by modulating TGF-β signaling. As described herein, any cell type can be used in which TGF-β signaling modulation affects its differentiation state. Such cells might be stem cells, stem-cell-like cells or differentiated cells. Examples of stem cells include totipotent stem cells, pluripotent stem cells, and multipotent stem cells. A number of adult, embryonic, and cord blood stem cells are known, including hematopoietic stem cells, pancreatic stem cells, mesenchymal stem cells, bone marrow stromal stem cells, adipose derived adult stem cells, olfactory stem cells, gastrointestinal stem cells, mammary gland stem cells, umbilical cord epithelial cells, amniotic membrane epithelial cells, and limbal epithelial progenitor cells. Differentiated cells might include epithelial cells, fibroblasts, myocytes, pancreatic β cells, blood cells, neurons, smooth muscle cells, fat cells, oligodendrocytes, alveolar cells, epidermal cells, and keratocytes.

To isolate cells or tissues from an animal subject, any suitable method may be used (including the differential dispase digestion and rapid adhesion methods described herein). As one example, a typical method of isolating keratocytes from an animal subject includes removing an anterior corneoscleral segment from the globe of the animal subject's eye by cutting near the limbus with Wescott's scissors or other appropriate cutting implement (see Kawakita et al., Invest Ophthalmol. Vis. Sci. 47:1918-1927, 2006 and Espana et al., Invest Ophthalmol. Vis. Sci. 46:4528-4535, 2005). A central cornea can be obtained with an 8.0 mm Hessburg-Barron trephine or other suitable trephine system and transferred to an appropriate medium (e.g., KSFM). After removing Descemet's membrane and the corneal epithelium by digestion with an appropriate protease (e.g., Dispase II for 16 h at 4° C.), the remaining corneal stroma is incubated at 37° C. for a suitable amount of time (e.g., 16 h) in medium (e.g., DMEM) containing collagenase and any other appropriate components for digestion (e.g., HEPES, gentamicin, amphotericin) on a suitable culture substrate or vessel (e.g., multi-well plate, plastic dish). Then, cells are resuspended in a suitable medium (e.g., KSFM), centrifuged to remove residual matrices, resuspended again, and seeded on an appropriate culture substrate or vessel (e.g., multi-well plate, plastic dish) in a suitable medium such as KSFM or DMEM containing ITS or 10% FBS. When this primary culture reaches approximately 80% confluence, cells are rendered into single cells by incubation in an appropriate solution (e.g., balanced salt solution (BSS) containing 0.25% trypsin/1 mM EDTA) at 37° C. for approximately 1 to 5 minutes, and the enzymatic reaction is stopped by adding soybean-trypsin inhibitor. After centrifuging (e.g., at 800×g for 5 minutes), the cells are resuspended in a suitable medium (e.g., KSFM) and cultured until use.

A typical method of isolating limbal epithelial progenitor cells includes first isolating a corneoscleral ring from a cornea (as described in He et al., Invest. Ophthalmol. Vis. Sci 47:151-157, 2005; Kawakita et al., Am. J. Pathol. 167:381-393, 2005). Then, limbal corneal epithelial sheets are isolated from the corneoscleral ring by digestion with a suitable protease (e.g., 10 mg/ml Dispase II in KSFM at 37° C. for 2 hours). Such digestion conditions preferentially remove superficial and suprabasal cells while retaining the most primitive progenitor cells (e.g., stem cells and stem-cell-like cells). On the other hands, dispase digestion at 4° C. for 16 hours preferentially removes the most primitive progenitor cells (e.g., stem cells and stem-cell-like cells) and mesenchymal niche cells (i.e., cells supporting stem cell function).

Alternatively, the limbal corneal epithelial cells can be isolated from the corneoscleral ring by treatment with cell dissociation buffer prior to culturing in an appropriate medium (e.g., SHEM and KSFM+S with or without 3T3 cells). The sheets are trypsinized and cultured on a suitable culture substrate or vessel (e.g., multi-well plate or plastic with or without 3T3 fibroblast feeder layers) in an appropriate medium (e.g., SHEM).

The use of differential dispase digestion based on temperature and duration also provides sheets of cells that are enriched in a desired cell type. For example, dispase digestion at higher temperatures for shorter periods of time provides sheets of cells (following mechanical separation) in which superficial and suprabasal cells have been depleted while not depleting the most primitive stem cells and stem-cell-like cells.

Methods of expanding animal cells (isolated or in tissues) ex vivo whose differentiation state is controllable by modulating TGF-β signaling as described herein include placing the cells in an ex vivo culture system including a culture vessel, a matrix, and a medium, wherein the medium is essentially free of AM and non-human mesenchymal feeder cells. In ex vivo culture systems described herein, any suitable vessel can be used. Examples of suitable vessels include traditional tissue culture substrates such as 6-, 24-, and 96-well plates, Petri dishes, flasks, bottles, plastic, and coverslips.

Typically, any culture media that enables the proliferation of stem cells or stem-cell-like cells while maintaining the “stemness” or stem cell qualities of the stem cells, and/or maintaining already differentiated cells in their current state type, are particularly useful. In some embodiments, a culture medium that inhibits TGF-β signaling in the cells is preferred.

In one embodiment, isolated animal cells or tissues are expanded in a culture system comprising a culture vessel, a matrix and a medium, wherein the medium is essentially free of amniotic membrane and non-human mesenchymal feeder cells, under conditions that downregulate TGF-β signaling in the cells to allow the cells to proliferate without undergoing a change in their differentiation state. Culture systems involving the use of non-human mesenchymal feeder layers can be undesirable because feeder layers have been shown to transmit xenogenic diseases to the cells being cultured. Cultures systems that use such feeder layers restrict further manipulation of the expanded cells. Thus, examples of suitable media for use in ex vivo culture systems include a medium essentially free of AM and non-human mesenchymal feeder cells. To downregulate TGF-β signaling in the cells, the cells can be cultured in a serum-free medium (e.g., KSFM) having less than about 10 ng/ml (e.g., less than 11, 10, 9, 8, 7, 6, 5, 4, 3, 2, 1, 0.5, 0.25, 0.1, 0.05, and 0.01 ng/ml) of TGF-β and less than about 0.10 mM (e.g., less than 0.11, 0.10, 0.09, 0.08, 0.07, 0.06, 0.05, 0.04, 0.03, 0.02, 0.01, and 0.005 mM) Ca²⁺. For culturing keratocytes, a typical medium is KSFM (cat. no. 17005-042, Gibco, Carlsbad, Calif.), a defined keratocyte serum-free medium that has a lower [Ca²⁺] (e.g., less than about 0.10 mM such as between about 0.03 and about 0.09 mM) than DMEM and that has no FBS (serum).

In alternative methods, limbal epithelial progenitor cells can be cultured in (a) KSFM, (b) seeded on AM derivatives and cultured in KSFM, (c) cultured in KSFM to which AM derivatives have been added, and (d) cultured in serum-containing medium to which AM derivatives have been added.

In some methods of an ex vivo culture systems in which TGF-β signaling is downregulated, the cells (isolated or in tissues) are cultured in a medium containing serum (and thus TGF-β) and a Ca²⁺ concentration up to about 1.8 mM Ca²⁺, conditions which normally upregulates TGF-β signaling, by adding an agent that downregulates TGF-β signaling in the cells. For example, instead of using KSFM for expanding animal cells, an alternative media such as SHEM (described in Meller et al., Br J Ophthalmol., 86:463-471, 2002; Grueterich and Tseng, Arch Ophthalmol., 120:783-790, 2002), which contains FBS and a high [Ca²⁺], that will upregulate TGF-β signaling. If SHEM is used, however, an agent that downregulates TGF-β signaling in the cells (e.g., AM derivatives) is added to the media.

In other embodiments, conditions which downregulate TGF-β signaling in the cells include seeding the cells at a cell density sufficiently low to prevent transient amplifying cells (TACs) from having a negative paracrine effect (e.g., secretion of TGF-β1 or TGFβ2) on the limbal epithelial progenitor cells and for a time period that is greater than that of the TACs. In such embodiments, the cell density is typically between about 10 and 500 cells/cm² and the time period is greater than about 3 weeks.

In the ex vivo culture systems and methods described herein, any suitable agent for downregulating TGF-62 signaling in a cell can be used. Examples of agents that downregulate TGF-β signaling include those that downregulate transcription of TGFβ gene in the cells. In some cases, the agent may specifically bind to TGFβ (e.g., an antibody), while in other cases, the agent may antagonize a receptor for TGFβ. Small molecule TGFβ signaling inhibitors such as SB-431542 (Hjelmand et al., Mol Cancer Ther. 3(6):737-745, 2004) and those described below might be used to downregulate TGFβ signaling in cells. A serine/threonine protein kinase inhibitor, a molecule that prevents translocation of a Smad protein from the cytoplasm of the cell to its nucleus, AM derivatives, AME, processed non-intact AM, and a purified component of AM (e.g., HA, HA-ITI, lumican, TSG-6, pentraxin and thrombospondin) are further examples of agents that can be used to downregulate TGFβ signaling in the cells at the transcriptional level. In some experiments described herein, limbal epithelial progenitor cells and keratocytes cultured in medium containing AM derivatives were expanded while maintaining their characteristic phenotypes.

A suitable form of isolated AM is described in U.S. Pat. Nos. 6,152,142, and 6,326,019. Processed AM might take the form of a powder (e.g., lyophilized and ground or pulverized AM) or other suitable form of AM. In addition, portions of AM might be used such as extracts of AM (see, e.g., U.S. provisional patent application 60/657,399) or purified components of AM such as extracellular matrix components such as HA, HA-ITI, and lumican (see, e.g., U.S. provisional patent application 60/657,399). Methods of culturing cells on AM (e.g., AM stromal matrix) in culture medium containing serum that prevent the differentiation of the cells are described herein.

A number of additional agents that downregulate TGF-β signaling are known and can be used in ex vivo culture systems and in methods described herein. Typical agents for modulating expression (and thus signaling) of intracellular proteins are mutants proteins, nucleic acids, and small organic or inorganic molecules. Examples of proteins that can modulate TGF-β expression and/or activity in a cell include variants or native TGF-β proteins or receptors thereof that can compete with a native TGF-β protein or receptor thereof. Such protein variants can be generated through various techniques known in the art. For example, protein variants can be made by mutagenesis, such as by introducing discrete point mutation(s), or by truncation. Mutation can give rise to a protein variant having substantially the same, or merely a subset of the functional activity of a native protein.

Another agent that can modulate TGF-β signaling is a TGF-β based or TGF-β receptor-based non-peptide mimetic or chemically modified form of a TGFβ or a TGF-β receptor that disrupts binding of between a TGF-β protein and its receptor. See, e.g., Freidinger et al. in Peptides: Chemistry and Biology, G. R. Marshall ed., ESCOM Publisher: Leiden, Netherlands, 1988). TGF-β proteins or receptors thereof may, for example, be chemically modified to create protein derivatives by forming covalent or aggregate conjugates with other chemical moieties, such as glycosyl groups, lipids, phosphate, acetyl groups and the like. Covalent derivatives of a protein can be prepared by linking the chemical moieties to functional groups on amino acid side chains of the protein or at the N-terminus or at the C-terminus of the polypeptide.

The agent that directly reduces TGF-β signaling can also be a nucleic acid that modulates expression of a TGF-β protein or receptor thereof. For example, the nucleic acid can be an antisense nucleic acid that hybridizes to mRNA encoding the TGFβ or receptor thereof. Antisense nucleic acid molecules for use herein are those that specifically hybridize (e.g. bind) under cellular conditions to cellular mRNA and/or genomic DNA encoding the protein of interest in a manner that inhibits expression of the protein, e.g., by inhibiting transcription and/or translation. Antisense constructs can be delivered using an expression vector plasmid or any other suitable means.

Ribozyme molecules designed to catalytically cleave TGF-β or TGFβ receptor mRNA transcripts can also be used to prevent translation of and expression of these proteins (see, e.g., PCT Publication No. WO 90/11364, published Oct. 4, 1990; Sarver et al., Science 247:1222-1225, 1990 and U.S. Pat. No. 5,093,246). In other embodiments, endogenous TGF-β or TGFβ receptor gene expression might be reduced by targeting deoxyribonucleotide sequences complementary to the regulatory region of the TGF-β or TGF-β receptor gene (i.e., the TGF-β or TGF-β receptor promoter) to form triple helical structures that prevent transcription of the targeted gene. (See generally, Helene, C., Anticancer Drug Des. 6(6):569-84, 1991; Helene, C., et al., Ann. N.Y. Acad. Sci. 660:27-36, 1992; and Maher, L. J., Bioassays 14(12):807-15, 1992). Inhibition of TGF-β gene expression might also be performed using RNA interference (RNAi) techniques. Such techniques are described in, for example, Zhou et al., Curr Top Med Chem, 6:901-911, 2006; Morris, K. V., BioTechniques April, Suppl:7-13, 2006; and Gilmore et al., Curr Drug Deliv. 3:147-150, 2006.

An example of a protein that can modulate TGF-β signaling is an antibody that specifically binds a TGF-β or a TGF-β receptor. Such an antibody can be used to interfere with the interaction of the TGF-β and its receptor or to directly antagonize the receptor.

### Ex Vivo Method of Expanding Limbal Epithelial Progenitor Cells

In another aspect, ex vivo method of expanding limbal epithelial progenitor cells (isolated or in tissues) in a cell culture are disclosed. In some embodiments, the cell culture is initiated with a mixture of limbal progenitor cells and transient amplifying cells (TACs). This method comprises placing the mixture of limbal epithelial progenitor cells and TACs in an ex vivo culture system including a culture vessel, a matrix, and a medium, and culturing the cells in the ex vivo culture system at a sufficiently low cell density for a time period exceeding the lifespan of the TACs under conditions suitable for expanding the limbal epithelial progenitor cells.

In a typical method, the mixture of limbal progenitor cells and TACs is obtained from donor (e.g., human) limbal corneal epithelial sheets isolated by digestion with an appropriate protease (e.g., Dispase II) in an appropriate medium (e.g., KSFM at 37° C. for 2 h) from the corneoscleral ring (see Espana et al., Invest. Ophthalmol. Vis. Sci. 44:4275-4281, 2003). This isolated mixture of limbal epithelial progenitor cells and TACs are seeded in an appropriate medium at a density of about 10-1,000 cells/cm² and cultured for a time period greater than about 3 weeks. In this method, the mixture of cells is seeded in the culture system at a cell density sufficiently low to prevent the TACs from having a negative paracrine effect in the limbal epithelial progenitor cells. The cells are incubated under appropriate conditions for cell expansion (e.g., at 37° C., in a 5% CO₂ humidified incubator, with medium changes as necessary). Expanded cells can be re-seeded into new culture vessels for further expansion. Once expanded to desired numbers, cells can be harvested for use. In an alternative embodiment, most TACs can be eliminated by isolating cells from the remaining limbal stroma that is surgically dissected and then digested by 2 mg/ml collagenase A solution in serum-free KSFM medium at 37° C. for 16 h (see Kawakita et al., Am J Pathol. 167:381-393, 2005) and cultured on plastic dishes in KSFM at a seeding density of approximately 10,000 cells/cm².

### Ex Vivo Method of Expanding Limbal Epithelial Progenitor Cells using p38 MAP Kinase Inhibitors

Another aspect includes methods of expanding animal cells (isolated or in tissues) ex vivo whose differentiation state is controllable by modulating a mitogen-activated protein kinase (MAPK), using a p38 MAP kinase inhibitor. Such a method includes culturing cells derived from limbal explants on a matrix in a culture medium comprising a p38 MAP kinase inhibitor in an amount effective to facilitate outgrowth of the epithelial sheet while inhibiting abnormal epidermal differentiation of the cells relative to the cells grown without p38 MAP kinase inhibitor.

Non-limiting examples of p38 MAP kinase inhibitors are provided in Table 1 below:

TABLE 1

| Compound: | Structure | Formula: | Number |
|---|---|---|---|
| SB 203580 | 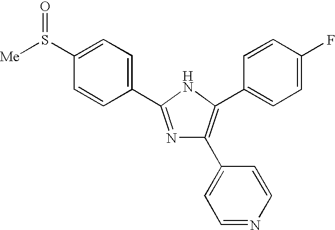 | C₂₁H₁₆FN₃OS | 152121-47-6 |
| SB 235699 | 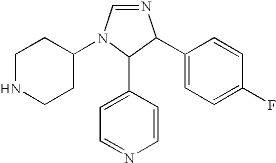 | C₁₉H₁₉FN₄ | 180869-32-3 |
| SB 220025 | 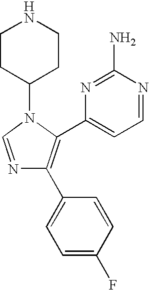 | C₁₈H₁₉FN₆ | 165806-53-1 |
| RWJ 68354 | 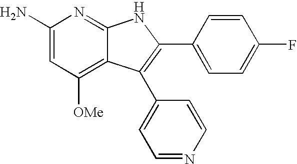 | C₁₉F₁₅FN₄O | 215306-39-1 |
| VX-745 | 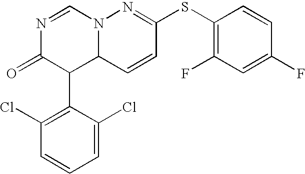 | C₁₉H₉Cl₂F₂N₃OS | 209410-46-8 |
| L-167307 | 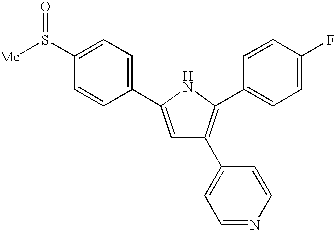 | C₂₂H₁₇FN₂OS | 188352-45-6 |
| SKF 86002 | 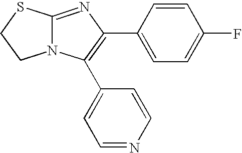 | C₁₆H₁₂FN₃S | 72873-74-6 |
| SB202190 | 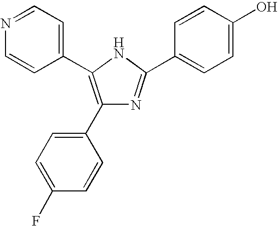 | C₂₀H₁₄FN₃O | 152121-30-7 |
| SB239063 | 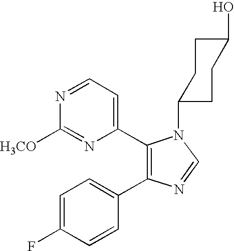 | C₂₀H₂₁FN₄O₂ | 193551-21-2 |
| P38 inhibitor 2-(4-Chloro- phenyl)-4-(4- fluoro- phenyl)-5- pyridin-4-yl- 1,2-dihydro- pyrazol-3-one | 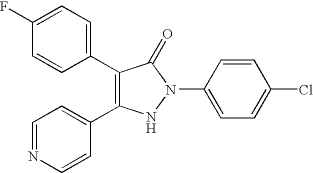 | C₂₀H₁₃ClFN₃O | 219138-24-6 |

In the ex vivo culture systems and methods described herein, any suitable agent for inhibiting p38 MAP kinase (MAPK) in a cell can be used. Examples of agents that inhibit the MAPK pathway include those that can prevent MAPK from phosphorylating transcription factors and cystolic targets in cells. In some cases, the agent may inhibit the catalytic activity of a kinase by competitive binding in the ATP pocket. The addition of a small molecular weight inhibitor of p38 MAP kinase, such as SB203580, L-167307, SKF 86002, SB 220025, SB 235699, VX-745, RWJ 68354, SB 202190, SB 239063, and 2-(4-chloro-phenyl)-4-(4-fluoro-phenyl)-5-pyridin-4-yl-1,2-dihydro-pyrazol-3-one, promotes the synthesis, deposition, formation, and ex vivo expansion of basement membrane components during epithelial outgrowth by limbal epithelial progenitor cells on denuded amniotic membrane (dAM).

In some methods of an ex vivo culture system in which p38 MAP kinase activity is modulated in the cells, the epithelial sheet is cultured on a collagen coated insert following an airlift method in SHEM medium with a p38 inhibitor, such as a pyridinylimidazole compound or a 2,4,5-triarylimidazole compound or SB203580, L-167307, SKF 86002, SB 220025, SB 235699, VX-745, RWJ 68354, SB 202190, SB 239063, and 2-(4-chloro-phenyl)-4-(4-fluoro-phenyl)-5-pyridin-4-yl-1,2-dihydro-pyrazol-3-one.

### Ex Vivo Expansion of Limbal Epithelial Cells and Mesenchymal Cells

In another aspect disclosed is a method for expanding limbal epithelial cells present in a limbal epithelial sheet. The method includes isolating an intact limbus from a biopsy of a donor eye of a living individual, an eye of a living, related individual, or from a cadaveric eye. The limbus obtained from the eye consists of the limbal epithelium and an underlying stroma. The limbus is then put in contact with a solution comprising Dispase II for a period of time and under conditions sufficient to substantially loosen a limbal epithelial sheet from the stroma, thereby forming a loosely adherent limbal epithelial sheet. The limbal epithelial sheet can then be mechanically separated from the underlying stroma, thereby isolating a substantially intact, viable, limbal epithelial sheet.

In a further embodiment of the disclosed method, the solution contacting the limbus accordingly comprises a substance chosen from either SHEM, a polyhydroxy alcohol, a sugar, or combinations thereof. Examples of polyhydroxy alcohol suitable for use in this embodiment include sorbitol, mannitol, and galactitol. In a further embodiment, the limbus/solution is maintained at a temperature from about 0° C. to about 37° C. for at least half an hour.

In a further embodiment of the disclosed method, the limbal epithelial sheet separated from the limbus is put in contact with the basement membrane side of an amniotic membrane. The limbal epithelial sheet and amniotic membrane thereby form a composite. This composite is then cultured for a period of time and under conditions sufficient to enable the epithelial cells to expand.

In another aspect, disclosed is a method of expanding mesenchymal cells ex vivo, while maintaining the phenotype of the mesenchymal cells. This method includes contacting the stromal side of the amniotic membrane with at least one type of mesenchymal cell, thereby forming a composite of the mesenchymal cells and the amniotic membrane. The composite is then cultured in a serum containing medium for a period of time and under conditions sufficient to enable the epithelial stem cells to expand.

### In Vitro Expansion of Limbal Epithelial Progenitor Cells using Human Amniotic Epithelial Cells as Feeder Layers

Another method for expanding limbal epithelial cells described herein is an in vitro expansion of animal cells comprising expanding such limbal epithelial cells on human amniotic epithelial cell feeder layers isolated from the amnion layer. Such animal cells include, but are not limited to, stem cells or stem-cell-like cells, such as limbal epithelial progenitor cells, umbilical cord epithelial cells, and amniotic membrane epithelial cells. Further, limbal epithelial progenitor cells can be obtained from corneoscleral tissue.

In some methods of in vitro expansion of animal cells, the human amniotic epithelial cells used as feeder layers for animal cells are mitomycin C (MMC)-treated human amniotic epithelial cells. In vitro proliferation of human amniotic epithelial cells (HAEC) in a SHEM culture medium containing 5% FBS together with EGF and insulin produces feeder layers that are more effective in promoting clonal growth of human amniotic epithelial cells. Limbal epithelial progenitor cells expanded on these HAEC feeder layers evidence more stem cell markers for longer periods of time than similar progenitor cells expanded on murine feeder layers.

### Treatment of Diseases

Animal cells expanded ex vivo according to systems and methods described herein can be transplanted into an animal subject suffering from any of a number of disease states in which stem cells or stem-cell-like cells are dysfunctional or lacking. For example, an animal subject suffering from HIV or cancer in which stem cells or stem-cell-like cells are dysfunctional can receive a transplantation of stem cells or stem-cell-like cells expanded ex vivo to restore the function of the dysfunctional stem cells or stem-cell-like cells. As another example, cells expanded according to the methods described above can be used to replace cells lost due to HIV infection or cancer or due to the side effects of treatment for those conditions (e.g., cell death caused by anti-viral or anti-neoplastic drugs or ionizing radiation).

A further aspect of this disclosure is surgical grafts, comprising cells or tissues cultured as described herein. In one embodiment, the surgical graft comprises an isolated, substantially intact, viable, limbal epithelial sheet or mesenchymal cells such as keratocytes. Surgical grafts can also be formed from expanded fetal mesenchymal cells, fibroblasts, endothelial cells, melanocytes, cartilage cells, bone cells, hematopoietic stem cells, bone marrow mesenchymal stem cells, adult mesenchymal stem cells or combinations thereof. The surgical graft may further comprise an amniotic membrane.

### EXAMPLES

By way of explanation, the examples set forth below describe methods for controlling the proliferation and differentiation of primary cells explanted from an animal or human subject such as corneal keratocytes and limbal epithelial progenitor cells from a variety of mammalian species including human beings.

### Example 1

### Preservation and Expansion of Primate Keratocyte Phenotype by Downregulating TGF-β Signaling in a Low Calcium Serum-Free Medium

Because TGF-β signaling is conserved among a large variety of different cell types, the results set forth below and methods described herein can be adapted for other cell types with minor modifications.

### Methods

Three rhesus monkeys (Macaca Mulatta), 4 years old, and rabbit and mouse cornea were obtained from an approved tissue-sharing program after euthanasia. An entire anterior corneoscleral segment was removed from the globe by cutting near the limbus with Wescott's scissors. A central cornea was obtained with an 8.0 mm Barron's trephine and immediately transferred to KSFM medium (cat# 17005-042, GIBCO Invitrogen corporation, Carlsbad, Calif.). After removing Descemet's membrane and the corneal endothelium, the corneal epithelium was removed by dispase digestion for 16 h at 4° C. and the remaining corneal stroma was incubated at 37° C. for 16 h in 2.5 ml of DMEM containing 1 mg/ml collagenase A, 20 mM HEPES, 50 μg/ml gentamicin and 1.25 μg/ml amphotericin in a plastic dish. Afterwards, corneal stromal cells were resuspended in 1 ml of KSFM, centrifuged to remove residual matrices, resuspended in KSFM, and seeded on plastic dishes in KSFM or DMEM containing insulin, transferrin, and selenium supplement (DMEM/ITSG) (cat# 41400-045, GIBCO, Carlsbad, Calif.) or 10% FBS (DMEM/10% FBS).

When the primary culture on plastic reached 80% confluence, cells were rendered into single cells by incubation in BSS containing 0.25% trypsin/1 mM EDTA at 37° C. for 1 to 5 minutes, and the enzymatic reaction was stopped by adding soybean-trypsin inhibitor. After centrifuging at 800×g for 5 minutes, cells were resuspended in KSFM, subdivided into 3 equal parts and seeded on plastic dishes. They were cultured in KSFM continuously until use. Keratocytes were similarly isolated from mouse, rabbit and human corneas and cultured in KSFM for comparison.

To verify cell proliferation in KSFM, primary cells in DMEM/ITS, DMEM/10% FBS and KSFM were subcultured at a density of 3,000 cells per 96-well plastic dish, and submitted at day 3 and day 7 to MTT assay (Promega Corporation, Madison, Wis.) according to the manufacturer's instructions. Using the culture medium alone as the negative control, this assay was validated by establishing a linear correlation between 2,500 and 10,000 passage 2 murine corneal fibroblasts. Cells at day 7 were also immunostained using an anti-Ki67 antibody (1:100). The number of Ki67 positive nuclei was randomly measured in 10 fields under high magnification (400×) for each culture, and the ratio of positive cells/total cells at each field was calculated. Experiments were performed in triplicate. Statistical analysis was performed by using Student's t-test. P<0.05 was considered statistically significant.

Freshly isolated cells expanded in KSFM were subcultured on plastic and upon 60-80% confluence cells at passage 1 (P1) were transferred to a dish in KSFM and continuously cultured and subcultured. As a comparison, cells were also subcultured on a dish in which the [Ca²⁺] was increased to 1.8 mM in KSFM by adding CaCl₂ with or without 10% FBS, or changed to DMEM/10% FBS to examine the ill effect of increasing Ca concentrations and/or addition of FBS. To determine the cell phenotype, cells were transfected for 24 h with self engineered aden-track-Kerapr3.2-intron-ECFP/BpA adenovirus at an MOI of 200 (see Kawakita et al., J Biol. Chem. 280:27085-27092, 2005). To examine the TGF-β signaling, the medium of P1 cells was replaced with fresh KSFM, DMEM/ITS, or DMEM/10% FBS 5 h before being transfected with replication-defective adenoviruses containing TGF-β1 or TGF-βRII promoters, each linked with luciferase (100 MOI) and containing CMV-β-galactosidae (30 MOI) for 48 hours. The promoter activity was measured by the Luciferase Assay System® (Promega, Madison, Wis.) and normalized with the β-galactosidase activity. In the same manner, TGF-βB1 and -β RII promoter activity of P1 cells was measured in cells cultured in KSFM, in which the [Ca²⁺] was increased to 1.8 mM (identical to DMEM) with or without 10% FBS. β-galactosidase activity was measured and relative transfection was normalized.

### Results

The cellular morphology of rhesus monkey keratocytes in vivo was studied by phase contrast microscopy and LIVE/DEAD assay®, of which the latter demarcated the entire cytoplasm. The monkey keratocytes showed a compact cell body with long dendritic cytoplasmic processes connecting with neighboring cells. These processes formed extensive intercellular contacts in a three dimensional pattern. In addition, CD34 was clearly expressed in the cytoplasm of these cells using both immunohistochemistry and immunofluorescence staining. Western blot analysis showed that an affinity-purified polyclonal antibody against human keratocan also cross-reacted with monkey keratocan. This cross-reactivity was attributed to the fact that there is 92.5 to 95% of homology between human and rhesus monkey keratocan genes. Western blot analysis showed a smear of high MW region in undigested samples consistent with the nature of proteoglycans, and a major band at 56 kDa in endo-β-galactosidase-digested monkey corneal stromal extracts. Using this antibody, keratocan was found to be expressed by keratocytes and the extracellular matrix in the entire monkey corneal stroma, but not by the corneal epithelium nor the corneal endothelium. These results showed that rhesus monkey keratocytes had a dendritic morphology and extensive cell-cell contacts, and expressed both CD34 and keratocan.

The monkey corneal stroma was subjected to collagenase digestion. The resultant cell suspension yielded approximately 1.5×10⁵ cells per cornea. Within 24 hours after seeding on plastic, cells attached well in DMEM/ITS, DMEM/10% FBS, or KSFM, but exhibited a distinctly different morphology. Cells cultured in DMEM/ITS for 7 days did not grow and showed a mixture of flattened and dendritic cells, while cells cultured in DMEM/10% FBS for 7 days reached confluence and showed a flattened fibroblastic morphology. In contrast, cells cultured in KSFM for 7 days had a higher cell density than DMEM/ITS and maintained a dendritic morphology.

To determine whether the dendritic morphology of keratocytes could be similarly maintained in KSFM in other species, primary cells were isolated from human, rabbit and mouse corneal stroma in the same manner and were cultured in KSFM on plastic for 48 hours. All cells showed prominent dendritic processes and extensive intercellular contacts similar to what was shown in monkey keratocytes. Both rabbit and human cells had a triangular cell body and longer dendrites; mouse cells had a rounder cell body and had thinner dendrites. These data showed that the dendritic morphology could be similarly maintained in KSFM for primary cultures of monkey, human, rabbit and mouse keratocytes.

To verify that cells were indeed proliferating in KSFM, an MTT assay was performed at day 3 and day 7 and immunostaining of Ki67 in primary cells at day 7. The number of cells measured by MTT was not significantly changed when cells were cultured in DMEM/ITS, but significantly increased when cultured in DMEM/10% FBS from day 3 to day 7 (p<0.01). The cell number in KSFM estimated by MTT was between that of DMEM/ITS and DMEM/10% FBS. (p<0.05, between day 3 and day 7). When assayed by the proportion of positive Ki67 nuclei, cellular proliferation in KSFM was also between that in DMEM/10% FBS and that in DMEM/ITS (p<0.05, both between KSFM and DMEM/10% FBS or DMEM/ITS). Cells maintained in DMEM/ITS could not be subcultured to P1. They immediately adopted a flattened morphology when subcultured in DMEM/10% FBS at P1. In contrast, cells subcultured in KSFM continued to maintain a dendritic morphology at P8 and P15. These results indicated that cells continued to maintain a dendritic morphology on plastic so long as cultured in KSFM. They reached the number of approximately 2.0×10⁵ in a 60 mm dish at each passage.

Because in vivo monkey keratocytes expressed keratocan and CD34, whether keratocan and CD34 proteins were continuously expressed by dendritic cells that were maintained at late passages in KSFM was examined, and whether such expression could be altered if the medium was switched to DMEM/ITS or DMEM/10% FBS was examined. When P14 cells cultured in KSFM were subcultured in DMEM/ITS or DMEM/10% FBS for 14 days, the dendritic morphology was changed to a flattened (fibroblastic) shape. In contrast, cells continuously subcultured in KSFM still maintained a dendritic morphology. Immunostaining revealed that expression of keratocan was markedly attenuated when subcultured in DMEM/ITS or DMEM/10% FBS, but continued in KSFM. Similarly, expression of CD34 was markedly downregulated when subcultured in DMEM/ITS or DMEM/10% FBS, but continued in KSFM. Because ALDH was a marker of human keratocytes, it was found that ALDH was expressed in primary cells cultured in DMEM/ITS, but lost in cells cultured in DMEM/10% FBS, but maintained in cells cultured in KSFM. Expression of ALDH was similarly downregulated when P14 keratocytes were subcultured in either DMEM/ITS or DMEM/10% FBS. These results indicated that the dendritic morphology of monkey keratocytes correlated well with expression of keratocan, CD34 and ALDH and such a phenotype could be maintained in KSFM, but lost when the medium was switched to either DMEM/10% FBS or DMEM/ITS.

KSFM is culture medium supplemented by growth factors including EGF and bFGF, and differs from DMEM-base medium in many aspects; the major features of KSFM are a low [Ca²⁺] and the lack of FBS. Whether high [Ca²⁺] or addition of 10% FBS or a combination of both might modulate the keratocyte phenotype determined by expression of keratocan was thus examined. To do so, the promoter activities following transient transfection of Aden-track-Kerapr3.2-intron-ECFP/BpA adenovirus containing CMV promoter-driven EGFP and keratocan promoter-driven ECFP in P1 cells was measured. In a given cell, expression of EGFP reflects the background transfection while expression of ECFP reflects the keratocan promoter activity. The protein expression of keratocan and CD34 was also monitored by immunostaining.

Compared to the dendritic morphology of keratocytes cultured in KSFM, most cells remained dendritic, but some cells became flattened in KSFM when [Ca²⁺] was increased to 1.8 mM. In contrast, the majority of cells lost the dendritic morphology and became flattened when 10% FBS was added in KSFM with low [Ca²⁺] or with high [Ca²⁺]. The percentage of ECFP-expressing cells to EGFP-expressing cells of the control cultured in KSFM alone was 70.3±9.2% (mean±s.d.). Such a percentage decreased to 62.0±9.6%, 33.3±5.4% and 29.8±4.5% when cells were cultured in KSFM with 1.8 mM [Ca²⁺], in KSFM with 10% FBS, and KSFM with 1.8 mM [Ca²⁺] and 10% FBS, respectively (p<0.01 for KSFM vs. KSFM+FBS or KSFM+[Ca²⁺]+FBS). There was no significant difference in those percentages between KSFM+[Ca²⁺]+FBS and DMEM/10% FBS nor between KSFM and KSFM+[Ca²⁺]. Immunostaining showed expression of keratocan in cells cultured in KSFM and in KSFM with high [Ca²⁺], but lost in KSFM with 10% FBS and in KSFM with high [Ca²⁺] and 10% FBS. Expression of CD34 was observed in cells cultured in KSFM and KSFM with high [Ca²⁺], but lost in KSFM with 10% FBS and in KSFM with high [Ca²⁺] and 10% FBS. These results indicated that the keratocyte phenotype was not significantly affected in KSFM by increasing [Ca²⁺], but was lost by addition of FBS. The latter detrimental effect was synergistic with increasing [Ca²⁺].

Whether TGF-β signaling was also similarly modulated by increasing [Ca²⁺] or addition of 10% FBS, or a combination of both in KSFM was examined by measuring the promoter activity of TGF-β1 and -β RII after transient adenoviral transfection. As compared to the control, i.e., cells cultured in DMEM/FBS 10% and adjusted by background transfection with CMV-β Gal, the promoter activity of TGF-β1 and -β RII was both significantly decreased in cells cultured in KSFM (p<0.05). There was no significant difference in the promoter activity between KSFM and DMEM/ITS. Compared to the control cultured in KSFM alone, increased [Ca²⁺] or addition of 10% FBS did not change the promoter activity for both TGF-β1 and -β RII (p>0.05). In contrast, a combination of increased [Ca²⁺] and addition of 10% FBS significantly upregulated the promoter activity for TGF-β1 and -β RII (p<0.05 and p<0.01, respectively). These results further supported the notion that the loss of keratocyte phenotype with respect to the dendritic morphology and expression of keratocan and CD34 as a result of increased [Ca²⁺] and addition of 10% FBS was correlated with upregulation of the transcriptional activity of TGF-β1 and -β RII genes.

To determine whether the aforementioned phenotype changes and suppression of transcription of TGF-β1 and TGF-β RII genes were correlated with change of Smad-mediated signaling, immunostaining of Smad2 and Smad4 was performed. The majority of cells cultured in DMEM/ITS or KSFM showed cytoplasmic localization of Smad2 and Smad4, while the majority of cells cultured in DMEM/10% FBS or KSFM with increased [Ca²⁺] and addition of 10% FBS showed nuclear localization of Smad2 and Smad4. The percentage of cells exhibiting nuclear accumulation of Smad2, an index suggestive of phosphorylation of Smad2, was 38±7.6% (mean±s.d.) in DMEM/ITS and 88.7±4.0% in DMEM/10% FBS, of which both were significantly higher than 19.3±5.1% in KSFM (p<0.01). Even when 4 ng/ml TGF-β1 was added in KSFM for 48 hours, the percentage of nuclear accumulation of Smad2 in cells increased to 34.7±4.9%, which was still not higher than that of DMEM/ITS (p>0.05). Similarly, the percentage of nuclear accumulation of Smad4 was 27.7±1.5%, 90.7±2.1%, and 12.0±3.0% in DMEM/ITS, DMEM/10% FBS, and KSFM, respectively. These results indicated that Smad-mediated TGF-β signaling was significantly downregulated in cells cultured in KSFM.

### Example 2

### Keratocan Expression of Murine Keratocytes is Maintained on AM by Downregulating TGF-β Signaling

Keratocytes display a dendritic morphology and express keratocan. When cultured using conventional methods, however, keratocytes lose their dendritic morphology and cease expression of keratocan. As described below, keratocytes were expanded on AM and examined to determine if they maintained their characteristic phenotype, including the expression of keratocan.

### Methods

Isolation and Culture of Keratocytes on Plastic or AM—Albino mice eyes were enucleated by forceps, washed profusely in PBS, and incubated in DMEM containing 20 mM HEPES, 15 mg/ml dispase II (Roche, Indianapolis, Ind.) and 100 mM sorbitol at 4° C. for 18 h (see Espana et al., Invest Ophthalmol Vis Sci. 44:4275-4281, 2003; Kawakita et al., Invest Ophthalmol Vis Sci. 45:3507-3512, 2004). The entire corneal epithelium loosened by this treatment was subsequently removed by vigorous shaking. Under a dissecting microscope, the corneal stroma was separated from the sclera at the corneoscleral limbus by pressing down the limbus with a 27 G needle while the eye was held with a forcep. Isolated corneal stromas were incubated overnight at 37° C. in DMEM containing 1.25 mg/ml collagenase A (Roche, Indianapolis, Ind.), 50 μg/ml gentamicin and 20 mM HEPES in a non-coated plastic dish until the tissue became “smeared” onto the dish bottom. Digested corneal stromas in collagenase A were centrifuged at 800×g for 5 min. Keratocytes were resuspended in DMEM containing 20 mM HEPES, ITS (5 μg/ml insulin, 5 μg/ml transferrin and 5 ng/ml sodium selenite), 50 μg/ml gentamicin and 1.25 μg/ml amphotericin B with or without 10% FBS. This keratocyte-containing cell suspension was then seeded on plastic dishes or on the stromal side of the AM (Bio-Tissue, Miami, Fla.) fastened to a culture insert as previously described (see Meller et al., Br J Ophthalmol 86, 463-471, 2002).

The suspension of keratocytes prepared from 3-4 murine corneal buttons was seeded on a 35 mm plastic dish or on the stromal surface of one 32 mm AM insert (see Espana et al., Invest Ophthalmol Vis Sci 44, 5136-5141, 2003). Cells were cultured in DMEM supplemented with 10% FBS (DMEM/10% FBS), and the medium was changed every 2-3 days. When cells reached 80-90% confluence, they were dissociated into single cells by incubation in 0.05% trypsin and 0.53 mM EDTA in HBSS at 37° C. for 5 min in plastic dishes or for 20 min in AM inserts, followed by vigorous pipetting. After centrifuging at 800×g for 5 min, cells were resuspended in DMEM/10% FBS and seeded on a plastic dish or AM stroma. They were cultured in DMEM containing 10% FBS, 20 mM HEPES, 50 μg/ml gentamicin and 1.25 μg/ml amphotericin B.

TGF-β1 Challenge and Neutralizing Antibody—To assess whether TGF-β1 affected the cell phenotype, triggered Smad 2 and Smad 4 nuclear translocation, and differentiated keratocytes into myofibroblasts, 10 ng/ml human recombinant TGF-β1 (Sigma, St Louis, Mo.) was added to serum-free DMEM/ITS cells for 3 h or 5 days when cells expanded on AM were passed to 24 well plastic dishes and AM inserts, respectively. In addition, primary keratocytes were seeded and cultured on AM or plastic for 3 days in DMEM/ITS or DMEM/10% FBS for 24 h, of which the latter was treated with or without 10 μg of a monoclonal antibody neutralizing TGF-β1, -β2, and -β3 (R&D Systems, Minneapolis, Minn.) per ml of DMEM medium for 48 h before adenoviral transfection.

Assays of Cell Proliferation—To verify that cells indeed proliferated on AM, the passage 2 (P2) cells that were continuously cultured on either AM or plastic were subcultured at a density of 10,000 cells per 24-well plastic dish in DMEM/ITS or DMEM/10% FBS or on AM in DMEM/10% FBS. Cells were terminated at day 3 and day 7 for MTT assay (Roche, Nutley, N.J.) according to the manufacturer's instruction. This assay measured by absorbance at 550 nm yielded a linear correlation for cell numbers above 2,500 cells using P2 murine corneal fibroblasts. Cells at day 7 were also immunostained using an anti-Ki67 antibody. The number of Ki67 positive nuclei was randomly measured in 10 fields under high magnification (400×) for each culture. Experiments were performed in triplicate.

Transient Transfection and TGFβ Promoter Assays—Freshly isolated cells expanded on AM were subcultured on plastic and AM inserts. Upon reaching 60-80% confluence, cells in each 24 well plate or AM insert were co-transfected with 1.0 μg/ml plasmid DNA containing TGF-β2 or TGF-α RII promoter-luciferase and 1.0 μg/ml pCMV/Sport/Bgal (Invitrogen, Carlsbad, Calif.) using GeneJammer® (Stratagene, LaJolla, Calif.) according to manufacturer's protocol.

### Results

In Vivo Morphology and Keratocan Expression of Murine Keratocytes—In CD-1 albino murine globes, there is a visible boundary that demarcates the limbus between the cornea and the sclera. Such a demarcation facilitated the surgical isolation of the corneal stroma. Following the removal of an intact sheet of the ocular surface epithelium from the globe by Dispase II, the corneal stroma was dissected from the adjacent sclera. Live/Dead Assay® revealed that an overwhelming majority of keratocytes were viable and exhibited a 3-dimensional dendritic morphology in the corneal stroma. Some dead cells were found in the cut edge of the excised stroma. Keratocytes in the stroma expressed keratocan as evidenced by positive staining with an affinity-purified antibody against mouse keratocan peptide. In contrast, corneal epithelial cells or endothelial cells were not stained. These results indicated that in vivo murine keratocytes also exhibited a characteristic dendritic morphology and specifically expressed keratocan.

In Vitro Morphology and Proliferation of Keratocytes Cultured on AM—Keratocytes were then isolated from the corneal stroma by collagenase digestion. An average of 5,000 cells was obtained per mouse cornea. Cells at a density of 3,000 per cm² were seeded on either plastic or AM in DMEM/10% FBS. Within 12 h after seeding, cells attached on either substrate, and exhibited a distinctly different morphology. Cells on plastic dishes were evenly distributed on the flat surface and adopted a spindle-shaped morphology with a broad stellate cytoplasm. They became confluent in 4 to 5 days. In contrast, cells on AM were dendritic or satellite in shape and had a triangular cell body and a scanty cytoplasm which formed extensive intercellular networks, and projected their dendritic processes in a 3-dimensional manner. They became confluent in 10 days.

Upon reaching 80-90% confluence, cells expanded on AM or plastic were trypsinized and continuously passaged onto the same type of substrate as used in the primary culture (P0). Cells subcultured on plastic at P1 became more flattened. In contrast, cells expanded on AM subcultures still maintained a dendritic morphology with pronounced intercellular contacts. They continuously preserved such a dendritic morphology until passage 8, when cells became senescent. Using MTT assay, cells of P2 plastic cultures in DMEM/ITS did not show an increase of cell number during the one week of culturing, while cells on plastic in DMEM/10% FBS rapidly expanded in number. Cells cultured on AM in DMEM/10% FBS were intermediate between that of the above two conditions (p<0.05 cf. DMEM/ITS and p<0.01 cf. DMEM/10% FBS). At day 7, the number of Ki67 positive nuclei in cells cultured on AM in DMEM/10% FBS was significantly more than that of cells on plastic in DMEM/ITS, but less than cells cultured on plastic in DMEM/10% FBS (both p<0.01). Collectively, these results confirmed that cells cultured on AM continued to proliferate and maintained a dendritic morphology in a FBS-containing medium.

Phenotypic Characterization of Cells Expanded on AM—To confirm that dendritic cells expanded on AM were indeed keratocytes and not myofibroblasts, immunostaining was performed for the expression of keratocan and α-SMA, respectively. For primary cultures (P0), a majority of dendritic cells cultured on plastic in DMEM/ITS for 5 days expressed keratocan but not α-SMA. In contrast, cells cultured on plastic in DMEM/10% FBS were not dendritic and did not express keratocan; instead some cells expressed α-SMA. However, dendritic cells expanded on AM in DMEM/10% FBS maintained keratocan expression, and did not express α-SMA. In DMEM/10% FBS, CD34 was not expressed by cells cultured on plastic, but expressed by cells cultured on AM. In contrast, fibronectin was expressed extracellularly and intracellularly by cells cultured on plastic, but not expressed by cells cultured on AM. These results collectively indicated that the keratocyte phenotype was maintained by AM.

To confirm transcript expression of keratocan, total RNAs were extracted from cells on plastic and AM, and subjected to RT-PCR. The results showed that keratocan transcript (of the size of 1065 bp) was expressed by cells cultured on plastic at passage 0, but lost at passage 1 and thereafter. In contrast, the keratocan transcript was continuously expressed in an abundant amount from passage 0 to passage 3 and up to passage 8 when cultured on AM.

To verify the keratocan protein expression, insoluble matrix proteins were extracted by 4 M guanidine HCl and subjected to Western blot analysis using an antibody against the core protein of keratocan. The sample from the normal murine corneal stroma, which was used as the positive control, showed a dense smearing in the high molecular weight region. Nevertheless, the same sample after digestion with endo-β-galactosidase showed a positive protein band of ˜50 kDa. The undigested sample of AM cultures at passages 6 and 8 showed a similar faint smearing in the same high molecular weight region. Both samples after digestion with endo-β-galactosidase showed a strong positive protein band of 50 kDa. A similar 50 kDa band was obtained from P2 cultures on AM, but not from P2 cultures on plastic using digestion by keratanase II. In contrast, there was no smearing in the undigested sample, nor was the protein band detected after digestion in plastic cultures at passage 1 and thereafter. The negative control of pure AM extract alone without any cultured cells did not contain any keratocan without or with endo-β-galactosidase digestion. Because keratocan expression was strongly observed in the extracellular matrix of in vivo murine corneas, conditioned media from P2 murine keratocyte cultures was also examined for keratocan expression. The results showed that the digested samples of the conditioned medium from AM culture, but not plastic cultures, showed a 50 kD band. Collectively, these data indicated that in DMEM/10% FBS cells expanded on AM, but not plastic, expressed keratocan in the matrix and the conditioned medium.

Transient and Sustained Suppression of Smad-dependent TGF-β Signaling in Keratocytes Cultured on AM −10 ng/ml TGF-β1 was added to both plastic and AM cultures containing DMEM/ITS and the Smad-mediated TGF-β signaling was examined by immunolocalization of Smad 2 and Smad 4. Cells on AM or plastic responded differently to exogenous addition of TGF-β1. Nuclear localization of Smad 4 was found in 16% of cells on plastic without TGF-β1, but increased to 67% and 85% of cells cultured on plastic after 3 h and 5 days of TGF-β1 challenge, respectively. A similar trend was noted for nuclear localization of Smad 2. In contrast, no cell cultured on AM showed nuclear localization of Smad 2 and Smad 4 at 3 h and 5 days of TGF-β1 challenge. These results indicated that TGF-β signaling mediated by Smad was continuously suppressed in cells cultured on AM even in the presence of 10% FBS.

To confirm that TGF-β was indeed responsible for Smad signaling in DMEM/10% FBS, a neutralizing antibody to three TGF-β isoforms was added to the plastic cultures. Nuclear translocation of Smad 4 was prevented. To demonstrate whether nuclear localization of Smad 4 also correlated with downstream of TGF-β signaling, α-SMA expression was quantified in parallel. Thirty-nine percent of cells cultured on plastic differentiated into α-SMA-expressing myofibroblasts, but no cell on AM expressed α-SMA even after 5 days of continuous stimulation with TGF-β1. Taken together, these results demonstrated that Smad-mediated TGF-β signaling was inhibited in cells cultured on AM and suppression of Smad-mediated TGF-β signaling correlated with prevention of cells from differentiating into myofibroblasts.

Inhibition of TGF-β2 and TGF-βRII Transcriptional Activity in Keratocytes Cultured on AM—To determine whether the aforementioned downregulation of TGF-β signaling was mediated by suppressing TGF-β genes at the transcriptional level, TGF-β2 and TGFβ-RII promoter activities were evaluated by transient transfection. As compared to cells cultured on plastic and adjusted by background transfection with CMV-β Gal, the promoter activity of TGF-β2 and TGF-β RII was decreased 4.1-fold and 2.6-fold, respectively, in cells cultured on AM (both p<0.001). These data suggest that down-regulation of TGF-β signaling was indeed mediated by suppressing TGF-β2 and TGF-β-RII genes at the transcriptional level for cells expanded on AM.

Suppression of TGF-β Signaling Maintained Keratocan Expression—To demonstrate a direct link between downregulation of TGF-β signaling and keratocan expression, 50 multiplicity of infectivity (M.O.I.) of Aden-track-Kerapr3.2-intron-ECFP/BpA was added to cells cultured on plastic in either DMEM/ITS or DMEM/10% FBS, of which the latter was further treated with or without an antibody to neutralize all three TGF-β isoforms. Transfection efficiency was revealed by EGFP (green fluorescence) driven by CMV in the same construct, while expression of keratocan promoter was revealed by ECFP (blue fluorescence) in the same cell. Cells retained the dendritic morphology after transfection in the positive control cultured on plastic in DMEM/ITS or on AM in DMEM/10% FBS. Cells also maintained a flattened bipolar morphology in the negative control cultured on plastic in DMEM/10% FBS. These results indicated that transfection itself did not alter their respective characteristic cell morphology. Interestingly, the fibroblastic morphology did not revert to a dendritic morphology after the addition of TGF-β neutralizing antibody for 2 days. The overall transfection efficiency was more than 80% in these experiments. Under such a high transfection rate, keratocan promoter-driven ECFP expression was observed in 30-40% of cells cultured on plastic in DMEM/ITS, 15-20% of cells cultured on AM in DMEM/10% FBS, but less than 2% of cells cultured on plastic in DMEM/10% FBS. These results corroborated with the aforementioned pattern of keratocan transcript and protein expression in these three cultures. ECFP expression was restored to 10-15% of cells cultured on plastic in DMEM/10% FBS when TGF-β neutralizing antibody was added. Collectively, these results indicated that TGF-β in 10% FBS was indeed responsible for the suppression of keratocan expression for cells cultured on plastic, and that keratocan expression by cells cultured on AM in 10% FBS was correlated with suppression of Smad-mediated TGF-β signaling.

### Example 3

### Clonal Initiation and Expansion of Murine Limbal Progenitor Cells in a Fibroblast-Free, Matrix-Free, and Serum-Free Niche

### Methods

A flat mount preparation of freshly isolated intact human limbal epithelial sheet showed that p63 -positive (p63 being an epithelium-specific transcription factor) basal cells are grouped in clusters, indicating that progenitor cells are intermiixed with TACs in the limbal basal epithelium. Because TACs are known to have a negative paracrine influence on limbal epithelial progenitor cell renewal, it was hypothesized that elimination of TAC's paracrine influence by seeding at a low density and prolonging the culturing time beyond TAC's life span (about 3 weeks) would improve clonal initiation and expansion of limbal epithelial progenitor cells. Single cells dissociated from isolated mouse corneal/limbal sheets by trypsin/EDTA were seeded at a density of 40 cells/cm² in a defined keratinocyte serum-free medium (KSFM) (Gibco-BRL, Carlsbad, Calif.) containing 0.07 mM (low) Ca²⁺ but supplemented with insulin, bFGF, EGF and cholera toxin.

When cells were seeded at a low density of 40 cells/cm², no cell growth was noted for the first 3 wks (i.e., within TAC life-span). On day 25 (>3 wks) there emerged an average of 2 to 3 large clones (stained by crystal violet) per 60 mm dish, a frequency similar to that seeded on mitomycin C-arrested 3T3 fibroblast feeder layers (which had more smaller clones). The large clone had a smooth perimeter and consisted of small epithelial cells, resembling “holoclone”, which has been used to denote epidermal stem cells and limbal progenitor cells. Expression of K12 keratin was negative in KSFM but positive in 3T3 fibroblast cultures, suggesting that KSFM is more ideal for maintaining stemness (stem cell characteristics) because cellular differentiation is less promoted than in 3T3 fibroblast cultures.

Elimination of TAC's paracrine influence by seeding at a low density and by prolonging the culturing time beyond TAC's life span (>3 wks) improved eliciting clonal initiation and expansion of limbal progenitor cells. Using this technique, clones were generated that could continually be expanded for more than 25 passages for a period of nearly two years (each passage spanning for one month). Two types of clonal growth (one fast and the other aborted) could be generated from a single cell derived from these holoclones by limiting dilutions in a 96 well culture plate. Several such single-cell generated clones were expanded, and each proved to be non-transformed. These single cell-generated clones could be cryopreserved and then recovered more than once.

During the early stage of the clonal growth (i.e., before Day 14) nearly all cells were uniformly small and round (<10 μm), negative for K12 keratin expression, and positive for p63 and K14 keratin expression. After Day 14, some cells (especially in the periphery) became enlarged, negative for p63 expression, and positive for K12 keratin and α-smooth muscle actin expression. Elevating extracellular calcium concentration ([Ca²⁺]) to 0.9 mM and/or adding 5% FBS caused the cells to become enlarged and squamous, to express K12 keratin, and lose expression of p63. These treatments also increased the level of TGF-β in the conditioned medium when TAC differentiation appeared.

The foregoing technique was also successfully used to isolate similar small cells from human limbal epithelial sheets, umbilical cord epithelium, and human amniotic epithelium. For human limbal epithelial progenitor cells, the same results were obtained using the above technique except that the cell seeding density could be as high as 2,500 cells/cm². An alternative method to eliminate most of the TACs in the example of expanding human limbal epithelial progenitor cells is to surgically dissect the remaining limbal stroma from the sclera after dispase digestion as stated above, and then to digest it by 2 mg/ml collagenase A solution in KSFM medium at 37° C. for 16 h (Kawakita et al., Am J Pathol. 167:381-393, 2005). Cells thus isolated were then cultured on plastic dishes in KSFM at a seeding density of 10,000 cells/cm². For human amniotic epithelial progenitor cells, the same results were obtained using the above technique with the exception that Ca²⁺ concentrations could be elevated as high as 1.0 mM. For all of the above epithelial cell cultures, adding 5% FBS and increasing the Ca²⁺ concentration to 1.8 mM caused rapid differentiation of these cells. By isolating limbal epithelial progenitor cells from deep within the stroma, TACs can be avoided, as TACs are not located deep within the stroma.

To show that the ability of KSFM, a low-calcium, serum-free medium, to promote limbal epithelial progenitor cell isolation and expansion resides in its ability to suppress TGF-β signaling, adenoviral vectors containing the promoters of TGF-β1, TGF-β2, TGF-β3, or TGF-βRII and the reporter gene luciferase were constructed and used to monitor the transcriptional activity of these four TGF-β genes in transiently transfected human limbal epithelial progenitor cells. Using this promoter assay, transcription of TGF-β1 and TGF-βRII was found to be markedly downregulated in human limbal epithelial cells when the cells were maintained in KSFM medium when compared to SHEM which contains 5% FBS and high calcium. Addition of 5% FBS to and elevation of [Ca²⁺] to 0.9 mM in KSFM, markedly upregulated TGF-β1 promoter activity to the same level as SHEM in human limbal epithelial progenitor cells and monkey.

These promoter activities were markedly elevated in DMEM with 10% FBS or SHEM (containing DMEM/F12 (1/1) and 5% FBS), both with high Ca⁺² and FBS, but significantly downregulated in KSFM. These promoter activities were significantly downregulated when AM extracts were added in SHEM, suggesting that the capability of AM extracts in suppressing TGF-β signaling can take place even in medium with high Ca⁺² and FBS.

In other experiments, clonally expanded murine epithelial progenitor cells were seeded at 500 cells/cm² (low), 5,000 cells/cm² (intermediate) and 50,000 cells/cm² (high) densities in KSFM. After 6 to 14 days in culture, most cells were small without expressing K12 keratin at the low density, but were large and expressed K12 keratin at the intermediate and high densities. Conditioned media was collected from these three cultures after 3 days of culturing in KSFM and subjected to a Bio-Plex machine (Bio-Rad, Hercules, Calif.) using Beadlyte® TGF-β1, β2, β3 detection system (Upstate, Waltham, Mass.). 4.3±0.5 ng/ml TGF-β1 and 5.1±0.4 ng/ml TGF-β2 (n=3) was detected only in the conditioned media of high density cultures seeded at 5,000 cells/cm² but not in those of low density cultures seeded at 50 or 500 cells/cm². No TGF-β3 was detected. Because the detection limit of this system is down to 0.1 ng/ml, the above data indicated that the levels of TGF-β1 or β2 in the low density cultures should be less than 0.1 ng/ml. Using the methods described above, umbilical cord epithelial cells were expanded ex vivo while maintaining their stem cell phenotype.

### Example 4

### Amniotic Epithelial Cells Help Maintain HA-Containing Stromal Matrix in Intact AM to Support Limbal Epithelial Progenitor Cell Renewal by Downregulating TGF-β Signaling

To determine whether amniotic epithelial cells on intact AM are pushed away or grown over by limbal epithelial cells migrating from the limbal explant, expanded limbal epithelial outgrowth was removed as a sheet from intact or denuded AM, respectively, by the method described in Espana et al., Invest Ophthalmol Vis Sci 44:4275-4281, 2003. Devitalized amniotic epithelial cells were present on intact AM, but absent on denuded AM and remaining stroma. A distinct basement membrane judged by a linear staining to collagen IV, laminin 5 and collagen VII was noted in the outgrowth on intact AM. In contrast, staining to collagen IV and laminin 5 was sporadic and diffuse, while that to collagen VII was negative in the outgrowth on denuded AM. The same result was confirmed in the remaining stroma when the epithelial sheet was removed. The remaining stromal matrix of intact AM was thicker than that of denuded AM (dAM) after expansion. These results indicate that amniotic epithelial cells, although devitalized, still play an active role in modulating epithelial basement membrane assembly.

Because HA is a major component of AM stromal matrix, the presence of HA in intact and denuded AM stromal matrix was examined. Using biotinylated HA binding protein (HABP) to immunolocalize HA, HA was observed to be better preserved in intact AM than denuded AM, suggesting that amniotic epithelial cells may partake in preventing the degradation of HA-containing AM stromal matrix, thus helping to maintain the stromal matrix. The thickness of denuded AM stroma was increased when human corneal fibroblasts were seeded on the stromal side of the AM.

A Western blot analysis showed that HA in AM stromal matrix was covalently linked with inter-α-trypsin inhibitor (ITI). The heavy chains of ITI entered the SDS gel only after HA was digested by hyaluronidase (HAse). The HA-ITI complex was not only present in the insoluble extracts (obtained by 1 M NaCl and 4M guanidine HCl, respectively) but also in soluble AM extract (obtained by homogenization in PBS). The covalent linkage of HA with ITI stabilizes high MW status of HA, preventing HA degradation to small MW in part because ITI is a natural inhibitor of HAse. In a related experiment, AM extracts were digested with or without 50 μg/ml HAse. The presence of ITI in these extracts and their interactions with HA were examined by Western blot. ITI was present in all extracts before HAse digestion, but there were extra bands appearing after digestion. These results suggest that ITI exists in AM in at least two forms: free and HA-bound. Additional western blots revealed that TSG-6, a component important for the formation of HA-ITI complex, was found in soluble fraction and in 4M guanidine HCl-extracted insoluble fraction. Pentraxin (PTX3), a component known to help HA crosslinking, was also found mostly in soluble extracts. Furthermore, thrombospondin, an anti-angiogenic component also known to help HA crosslinking, was found in soluble extracts and 4M guanidine HCl extracts.

### Example 5

### In Vitro Method of Expanding Progenitor Cells on Human Amniotic Epithelial Cell (HAECs) Feeder Layers

Described herein are methods of expanding animal cells in vitro by seeding such cells on feeder layers. Such a method includes several steps. The first step includes isolating HAECs from human placenta. An intact layer of the amnion was mechanically peeled off of the placenta and individual HAECs are isolated from the amnion layer. The next step is to seed individual progenitor cells on the HAECs and to incubate the cell cultures in medium.

As described herein, the cells seeded on the HAECs can be any cell type which can differentiate. Such cells might be stem cells or differentiated cells. Examples of stem cells include totipotent stem cells, pluripotent stem cells, and multipotent stem cells. Differentiated cells might include epithelial cells, fibroblasts, myocytes, pancreatic β cells, blood cells, neurons, smooth muscle cells, fat cells, oligodendrocytes, alveolar cells, epidermal cells, and keratocytes.

To isolate HAECs, any suitable method may be used. As one example, a typical method of isolating HAECs includes peeling the amnion off from the chorion after the placenta has been washed to remove blood. Once the amnion is removed, 5×5 cm² pieces are cut and rinsed in HBSS. The amnion is then digested with an appropriate protease (e.g., Dispase II at 37° C. for 15 min), and incubated in the appropriate medium (e.g., KSFM) to generate a loose single layer of the amniotic epithelium, which can then be separated from the underlying stroma by gentle stripping off with jewelry forceps. Cells are rendered into single cells by incubation in an appropriate solution (e.g., 0.25% Trypsin/1 Mm EDTA at 37° C.). After centrifuging (e.g., at 2000 rpm for 5 minutes) cells are collected and viability determined.

Besides what has been mentioned above, another method of isolating limbal epithelial progenitor cells includes isolating limbal epithelial sheets directly from corneoscleral rims obtained immediately after penetrating keratoplasty based on a modified digestion using the appropriate protease (e.g., 10 mg/ml Dispase II in SHEM at 4° C. for 16 h). Isolated limbal epithelial sheets are rendered into single cells by digestion (e.g., 0.05% trypisn/0.53, Mm EDTA at 37° C. for 15 min), and then seeded in triplicate at a density of 50 cells/cm² on HAEC feeder layers. Cells are then incubated at 37° C. under 5% CO₂ and 95% humidity, and the medium was changed every 2-3 days.

### Methods

Human placentas were washed twice in HBSS without calcium or magnesium for 5 min each to remove the blood. An intact layer of the amnion was mechanically peeled off from the chorion, cut into pieces of approximately 5×5 cm², and rinsed in HBSS for another 5 min. The amnion was then incubated in with 10 mg/ml Dispase II in KSFM at 37° C. for 15 min to generate a loose single layer of the amniotic epithelium, which was then separated from the underlying stroma by gentle stripping off with jewlry forceps. Isolated epithelial sheets were further digested with 0.25% Trypsin/1 mM EDTA at 37° C. for 15 min; dissociated cells were collected after centrifugation at 2,000 rpm for 5 min. Cell viability was determined by exclusion of trypan blue dye and counted with a hemocytometer.

Single HAECs isolated from the amnion were plated on 100-mm-diameter culture dish at a density of 1.27×10⁴ cells per cm² (1 million cells/dish) in 9 different media made of KSFM, DMEM/F12, or SHEM. For both KSFM and DMEM/F12, additional modifications were made by adding calcium chloride and/or FBS. All experiments were performed in triplicate per condition. After reaching subconfluence, cells were digested with Trypsin/EDTA and subcultured at a 1:3 split in a given medium. For characterization of cell phenotype, cells from each condition were also grown in 8-chamber slides for immunostaining.

In order to study how cell proliferation might be affected by different media, the first passage of HAECs cultured in SHEM was subsequently subcultured in each of the above-mentioned 9 media and subjected to MTT assay, which is based on the cleavage of yellow tetrazolium salt MTT to purple formazan crystals by metabolically-active cells (see Kamiya et al., Exp Eye Res. 80:671-679, 2005). After cells were incubated for 4 h, formation of formazan dye in the microtiterplate was spectrophotometrically quantified with an ELISA plate reader set at 570 nm. Experiments were conducted using 5 samples per condition.

In order to examine whether HAECs could support clonal growth of human limbal progenitor cells, clonal assay was performed using a method reported by Rheinwald and Green (see Rheinwald & Green, 6:331-337, 1975) and modified by us for limbal epithelial cells (see Tseng et al., Curr Eye Res. 15:973-984, 1996). In brief, both NIH 3T3 fibroblasts grown in DMEM containing 10% newborne calf serum at 80% subconfluence, and the 8^{th} passage of HAECs grown in SHEM were treated with 4 μg/ml mitomycin C for 2 h and then trypsinized and plated at a density of 2×10⁴ cells/cm² in 60-mm dishes. Human limbal epithelial sheets were isolated directly from corneoscleral rims obtained immediately after penetrating keratoplasty based on our modified digestion method using 10 mg/ml Dispase 2 in SHEM at 4° C. for 16 h (see Espana et al., Ophthalmol Vis Sci. 44:4275-4281, 2003). Isolated limbal epithelial sheets were rendered into single cells by digestion with 0.05% trypsin/0.53 mM EDTA at 37° C. for 15 min, and seeded in triplicate at a density of 50 cells/cm² (500 cells/well) on either HAEC or 3T3 fibroblast feeder layers in six-well culture plates containing SHEM. Cultures were incubated at 37° C. under 5% CO₂ and 95% humidity, and the medium was changed every 2-3 days. Colonies were counted at Day 6 and Day 10, followed by fixation in cold methanol at Day 10 or Day 14, stained with crystal violet dye, and photographed.

The colony-forming efficiency (CFE) was calculated as the number of colonies per well divided by 500 (i.e., the total seeded cells per well). Cell sizes of colonies were determined by an imaging software using AlphaImage 2200 (version 3.2.1, AlphaInnotech, San Leandro, Calif.) by selecting 3 clones with the size of at least 2-2.5 mm in diameter from each feeder layer. Because the homogeneity of cell size in HAEC-carried clones and the heterogeneity of 3T3-carried clones, 10 randomly chosen fields at 400× magnification were taken for each selected HAEC-supported clones to compared with 5 randomly chosen fields were taken from the periphery and the center of 3T3-supported clones, respectively.

Human amnion was embedded in OCT, snapped frozen in liquid nitrogen, and cut into 5 μm-thick sections. Both tissue sections and HAECs cultured on plastic were subjected to immunostaining with appropriate dilutions of primary antibody summarized in and their respective secondary antibodies. Briefly, each sample was fixated in cold methanol for 10 min at −20° C., permeabilized in 0.2% Triton X-100 for 15 min and blocked with 2% bovine serum albumin for 30 min at room temperature. Cells were then incubated with the primary antibody for 16 h at 4° C. After 3 times of wash with PBS, specific binding was detected by a FITC-conjugated anti-mouse or anti-rabbit secondary antibody incubated for 30 min at room temperature. Finally, the sample was counterstained with Hoescht 33342 and mounted in anti-fading solution (Vector Labs, Burlingame, Calif., USA). For Ki67, we used immunoperoxidase protocol (ABC kit Vectastain Elite, Vector Labs) followed by a DAB kit (Dako, Carpinteria, Calif.), and counterstained the sample with hematoxylin. The negative control was performed by substitution of the primary antibody with PBS. For double immunostaining of both pan-cytokeratins and vimentin, tissue sections or cells were incubated with respective primary antibodies followed by sequential incubation for 30 min of AEC kit (Sigma, St. Louis, Mo.) and TRITC-conjugated anti-goat secondary antibody, respectively. Images were photographed with a NikonTe-2000u Eclipse epi-fluorescent microscope (Nikon, Tokyo, Japan). Positive immunostaining was counted at 200× magnification at 10 random fields in each sample.

HAECs isolated immediately from amniotic epithelial sheets as well as from cultures of Passages 0, 1, and 6 in SHEM were quantitatively compared for their cytoskeleton protein expression using Western blots. Cultured cells were collected and solubilized in the lysis buffer containing 50 mM Tris-HCl (PH 8.0), 1% NP-40, 0.5% DOC, 0.1% SDS, 150 mM NaCl, 5 mM EDTA, 1 mM PMSF and a protease inhibitor cocktail. Total cell proteins were extracted from fresh amniotic sheets by gentle scraping with the same lysis buffer. Proteins in these lysates were denatured by boiling for 5 min with the equal volume of 2× Tris-glycine SDS sample buffer, separated by 4% to 15% gradient SDS-PAGE, and transferred to nitrocellulose membranes. Five percent nonfat milk was incubated for 1 h to block non-specific binding, followed by each of the three primary antibodies to vimentin, cytokeratin 18 (both at 1:500), and β-actin (at 1:5000). The specific binding was then detected by respectively anti-mouse and anti-rabbit horseradish peroxidase (HRP)-conjugated antibodies (at 1:1000), and visualized by enhanced chemiluminescence method.

### Results

Limbal epithelial progenitor cells form clones in HAEC feeder layers. In general, clones grown on HAEC feeder layers emerged slower, i.e., after at least 5 days, and were fewer and smaller. In contrast, limbal cells grown on murine 3T3 feeder layers emerged as early as day 3, and were more and larger. The border of limbal clones on 3T3 feeder layer was smooth, presumably because 3T3 fibroblasts were pushed by expanding limbal epithelial cells. Interestingly, the border of limbal clones on HAEC was not smooth because the entire clone was growing on the top of their underlying feeder cells. The CFE on the HAEC feeder layer was 1.1±0.4% on Day 6 and 2.4±0.7% on day 10, which were significantly less than 7.2±1.2% (p=0.007, n=3) and 11.1±2.1% (p=0.011, n=3) on their 3T3 counterparts, respectively. Under low magnification, limbal clones supported by HAEC feeder layers were uniformly compact and the border was irregular. In contrast, clones supported by 3T3 feeder layers had less cellularity in the center of the clone and were round with a smooth border. Under high magnification, HAEC-supported limbal clones consisted of uniformly small and oval epithelial cells. In contrast, 3T3-supported clones were composed of cells with variable sizes with those in the center being large and squamous. Indeed, the average cell size for HAEC-supported clones was 19.8±2.2 μm, which was much smaller than 24.4±2.0 μm of the periphery (p=0.028, n=3) and 44.2±2.2 μm of the center (p=0.017, n=3) of 3T3-supported clones.

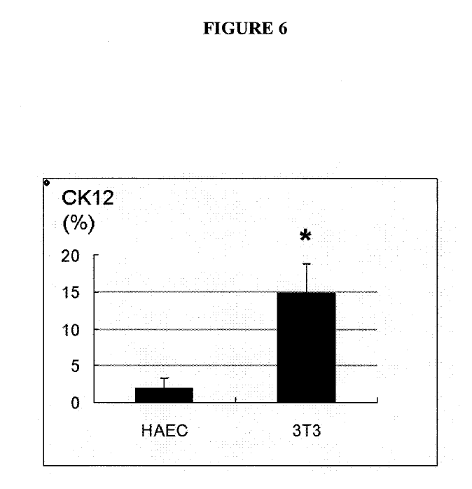

Besides differences in the cell size, expression of differentiated cell markers was different in these two types of clones. A corneal epithelium-specific marker, CK12, was not expressed by the amniotic epithelium, but expressed by human limbal suprabasal cells. Except for few single cells in the center of the clone, expression of CK12 was nearly totally negative in HAEC-supported clones. On the contrary, many cells vividly expressed CK12 on 3T3-supported clones. The percentage of CK12-postive cells, indicative of the extent of corneal epithelial differentiation, was significantly higher in 3T3-supported clones than that in HAEC-supported clones (14.8±5.0% versus 1.8±1.6% ) (p=0.019, n=3) (see FIG. 6).

Connexin 43, a membrane protein critical for gap junctional communication, is also found to be expressed by suprabasal cell layers of the human limbal epithelium. As expected, we also noted a punctate staining pattern of connexin 43 at intercellular junctions in the suprabasal layers of the human limbal epithelium. Interestingly, cells in HAEC-supported clones were entirely negative to the connexin 43 staining. In contrast, cells from the mid-periphery and the center of 3T3-supported clones revealed positive Cx43 staining at intercellular junctions.

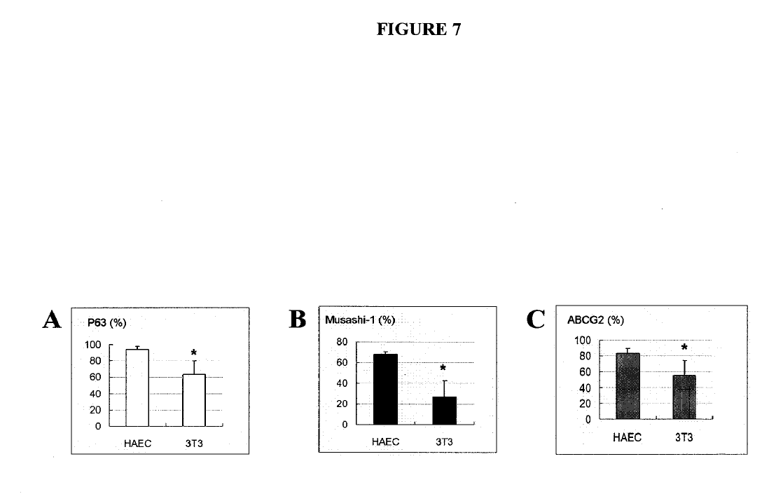

Having demonstrated the phenotype of HAEC-supported clones was less differentiated than that of 3T3-supported clones, it was also determined whether HAEC-supported clones actually expressed such stem cell markers as p63, Musashi-1, and ABCG2 proteins. Expression of p63 is found to be in epidermal and limbal epithelial progenitor cells. On HAEC feeder layers, positive nuclear p63 immunostaining was found in nearly all cells in the entire clone except for very few cells in the center of clone. On 3T3 feeder layers, positive nuclear p63 staining was restricted to peripheral small cells, and large squamous cells in the center of the clone were negative. The percentage of p63-positive cells in HAEC-supported clones was 93.7±4.4%, which was significantly higher than 63.5±16.3% in on 3T3-supported clones (p=0.030, n=4) (see FIG. 7A). Musashi-1, a neural stem cell marker, was found in the nucleus of the subventrical zone of the mouse brain. In the human limbus, strong nuclear staining of Musashi-1 was found in basal epithelial cells with a faint staining noted throughout the whole epithelium. Nuclear expression of Musashi-1 was found in most cells in HAEC-supported clones. In contrast, there was a gradient of positive nuclear staining from the periphery to the center of 3T3-supported clones. The percentage of Musashi-1-positive cells in HAEC-supported clones was 68.3±2.6%, which was significantly higher than 26.8±15.8% in 3T3-supported clones (p=0.012, n=4) (see FIG. 7B)

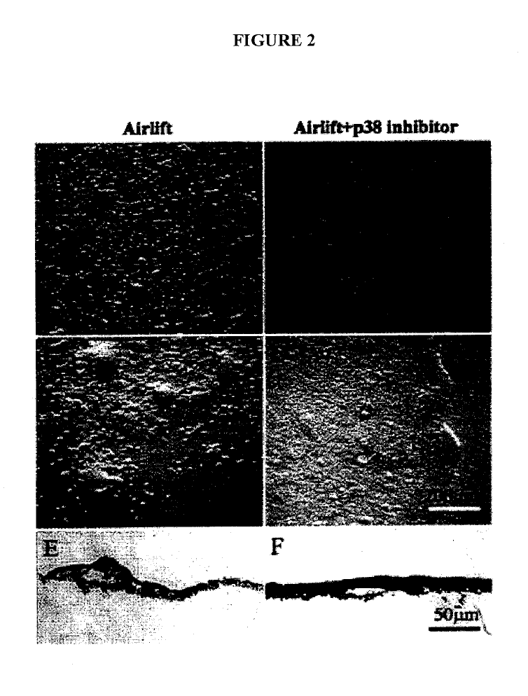

Expression of ATP-binding cassette subfamily G2 (ABCG2), a plasma membrane transporter, is highly conserved in primitive stem cells from a variety of tissue sources. Previously, expression of ABCG2 was also found to be predominantly by limbal basal epithelial cells. As expected, we also noted such a positive expression in the normal limbal tissue. In HAEC-supported clones, the majority of cells were positively stained to ABCG2, forming patches intermixed with few negative cells throughout the entire clone. Contrary to such an evenly distributed pattern, positive expression of ABCG2 was mainly observed in clusters of small cells in the periphery of 3T3-supported clones; cells in the center of the clone were negative. The percentage of ABCG2-positive cells in HAEC-supported clones was 87.5±6.3%, which was significantly higher than 54.9±19.1% in 3T3-supported clones (p=0.036, n=4) (See FIG. 2C). Taken together, the data indicated that HAEC-supported clones express significantly more stem cell-associated markers than 3T3-supported clones, indicative of preservation of more epithelial stem cells.

### Example 6

### Addition of TGF-β1 Induced Expression of α-Smooth Muscle Actin (α-SMA) in Murine Epithelial Progenitor Cells Via Activation of Both Smad and β-catenin/LEF-1 Signaling Pathways

In clonal cultures of murine limbal epithelial progenitor cells, expression of α-SMA, a marker for myofibroblasts, was not detected when clonally expanded murine limbal epithelial cells were seeded at low density (500 cells/cm²), but increasingly detected when cells were seeded at the intermediate (5,000 cells/cm²) and high seeding densities (50,000 cells/cm²). Cells expressing α-SMA were larger and squamous and could also express p63 in the nucleus. Addition of 5 ng/ml TGF-β1 to KSFM upregulated α-SMA expression in cultures seeded at the low density. In contrast, addition of 10 μg/ml of a neutralizing antibody against three TGF-β isoforms significantly reduced α-SMA expression in cultures seeded at the high density. α-SMA was not expressed in the center, but was expressed in the periphery of the single cell-derived clone after being expanded more than 3 weeks. Under these conditions, expression of S100A shifted from cytoplasm to the nucleus, signaling of Smad 4 moved from cytoplasm to the nucleus, expression of E-catenin moved from the intercellular junction to the cytoplasm and the perinucleus, the signaling of β-catenin moved from the intercellular junctions to the nucleus), and the signaling of LEF-1 also moved from the cytoplasm to the nucleus. These results suggest that the irreversible epithelial-mesenchymal transition can take place in limbal epithelial progenitor cells by expression of α-SMA and S100A4 and is correlated with activation of Smad-mediated signaling and β-catenin/LEF-1 mediated signaling.

### Example 7

### A TGF-β Promoter Assay that Demonstrates the Suppressing Effect of TGF-β Signaling by AM Derivatives in Both Human Limbal Epithelial Progenitor Cells and Human Corneal Fibroblasts

Human corneal fibroblasts were transiently transfected with the aforementioned adenoviral promoter constructs. In these cells, TGF-β1 promoter activity was significantly suppressed by an AME prepared according to the method described in U.S. provisional patent application filed Mar. 2, 2005 and entitled “Suppression Of TGF-B Activity By Amniotic Membrane Extracts, Compositions Thereof, And Methods For The Prevention And Suppression Of Scarring And Inflammation” in a dose-dependent manner (from 0.04 to 125 μg/ml) in human corneal fibroblasts. In human limbal epithelial cells, TGF-β1 promoter activity was significantly suppressed by 25 μg/ml AME in both KSFM and SHEM media. Furthermore, the suppressive activity of AME (which contained ˜0.8 μg/ml HA) was more potent than 125 mg/ml high MW pure HA alone. The suppressive effect of both AME and HA alone was lost after pretreatment with HAse. No suppressive effect was noted in the control when HAse alone was added together with BSA. A similar result was obtained for TGF-βRII promoter activity.

Single cell expanded murine limbal epithelial cells were seeded at 50 cells/cm² in KSFM and cultured for 6 days. Addition of TGF-β1 at 10 pg/ml and 150 pg/ml dose-dependently suppressed the clonal expansion. In contrast, addition of 10 μg/ml neutralizing antibody against three TGF-β isoforms to the control to suppress endogenous production of TGF-β resulted in expansion of clonal growth with pronounced cell migration. Cells seeded at 20,000 cells/cm² in KSFM became enlarged. However, addition of 125 μg/ml AME promoted expansion of more small cells.

### Example 8

### Signaling Transduction Pathways Required for Ex Vivo Expansion of Human Limbal Explants on Intact AM

The results described below show that ex vivo expansion of human limbal epithelial progenitor cells on intact AM is mediated by the survival signaling pathway mediated by PI3K-Akt-FKHRL1 and the mitogenic MAPK pathway mediated by p44/42, but not by p38 and JNK.

### Methods

Human AM was provided by Bio-Tissue (Miami, Fla.) and stored at −80° C. before use. AM was devitalized by freezing and thawing and washed three times with HBSS before being fastened onto a 30 mm culture insert, Millicel-CM (which generated an insert with 23 mm diameter covered by AM), and placed in a 6 well plate (Meller, D. and Tseng S C G, Invest Ophthalmol Vis Sci., 40:878-86, 1999).

After removal of excessive sclera, iris, corneal endothelium, conjunctiva, and Tenon's capsule, the limbal ring was separated by a 7.5 mm trephine from donor human corneas. Each limbal ring was rinsed 3 times with SHEM media. The limbal ring was then exposed for 10 min to 1.2 units/ml Dispase II in Mg²⁺- and Ca²⁺-free HBSS at 37° C. under 95% humidity and 5% CO₂. Following three rinses with SHEM medium, each limbal ring was subdivided into two halves and each half further subdivided into 6 pieces of 1×1.5×2.5 mm explants. To eliminate variations of age, sex, and race, explants from the corresponding position of the same donor cornea were selected for the control and the experimental group, respectively. An explant was placed on the center of intact AM or plastic with the epithelial side facing up and cultured in SHEM medium. The experimental group was added with the inhibitor of desired concentration, while the control group was added with the same concentration of DMSO as the vehicle which was used to dissolve each inhibitor. The culture was maintained at 37° C. under 95% humidity and 5% CO₂, the medium was changed every other day, and their outgrowth was monitored daily for 17 days using an inverted phase microscope (Nikon, Japan). The outgrowth area was digitized every other day by Adobe Photoshop 5.5 and analyzed by NIH ImageJ 1.30v (NIH, Bethesda, Md.).

All experiments were performed at least in triplicate. Summary data were reported as means±S.D., compiled and analyzed by MicroSoft Excel™ (MicroSoft, Redmont, Wash.). The mean and standard deviation were calculated for each group using the appropriate version of Student's unpaired t-test. Test results were reported as two-tailed p values, where p<0.05 was considered statistically significant.

### Results

To ensure that the control without treatment had a consistent growth rate and pattern, a total of 33 limbal explants from 11 donors ranging 37 to 61 years old were examined. Under microscopic observation, it was noted that epithelial cells started to migrate from the limbal edge to AM in 28 of 33 explants (85%) at day 3-4, while from the corneal or scleral edge in the rest. At day 5, cell outgrowth could be discerned by the naked eye. The surface area was scanned and digitized every other day until day 17 when the outgrowth reached ˜80% confluence, i.e., ˜340 mm² of 415 mm² of the AM insert. The culture was terminated before reaching confluence to avoid possible underestimation caused by cell contact inhibition. The outgrowth rate of the control showed a consistent pattern as a group. The outgrowth rate was gradually increased from day 5 to day 9, but rapidly increased from day 9 to day 13, and gradually slowed down from day 13 to day 17.

The PI3K-Akt pathway controls cell survival, and inhibition of this pathway frequently leads to apopotosis. LY294002 is a specific inhibitor of PI3K, and one of the downstream target of PI3K is to phosphorylate and activate Akt kinase. The epithelial outgrowth was not significantly inhibited by 5 and 10 μM of LY294002 (p=0.85 and 0.09, respectively), but was significantly or completely inhibited by 20 and 50 μM of LY294002 (p=0.0008 and 0.0007, respectively). As compared to the control, complete inhibition of epithelial outgrowth was noted at 20 μM and 50 μM of LY294002. Addition of 10 μM of SR13668, a potent phosphor-Akt inhibitor, resulted in 50% reduction of the outgrowth rate from day 5 to day 11, and 60% reduction from then on. Addition of 50 μM of SR13668 completely inhibited the epithelial outgrowth. These results indicated that inhibition of either PI3K or Akt could completely abolish epithelial outgrowth from the limbal explant cultured on AM.

U0126 is a specific inhibitor of MAPK kinase MEK1/2. At 10 μM, it completely inhibited p44/42 MAPK phosphorylation in many cells. When 10 μM of U0126 was added, epithelial outgrowth was noted at day 8, which was significantly more delayed than the control. From day 13 on, the outgrowth from the U0126-treated explants was almost halted. At day 17, the average outgrowth area of the control and U0126-treated group was 334±34.3 mm² and 17.0±3.0 mm², respectively (p=0.0077). SB203580 and JNK inhibitor 1 are specific inhibitors for MAPK p38 kinase and JNK kinase, respectively. Addition of 10 μM of either SB203580 or JNK inhibitor 1 did not change the outgrowth which started at day 5 and reached similar rates when compared to the control. There appeared some promotion of epithelial outgrowth in SB203580-treated explants, but the difference did not reach a statistical difference (p=0.89). Collectively, these results indicated that inhibition of p44/42 MAPK, but not p38 kinases or JNK, of the MAPK family also completely abolished epithelial outgrowth from the limbal explant cultured on AM.

Because addition of LY294002, SR13668, or U0126 led to complete or significant inhibition of ex vivo expansion of limbal epithelial cells, these inhibitors were thus removed after the explants was treated with 50 μM LY294002, 50 μM SR13668, or 10 μM U0126 for 17 days, respectively. It was noted that the inhibition of epithelial outgrowth was reversible because the outgrowth re-initiated in 2 days after the culture medium containing the inhibitor was switched to the fresh medium. However, the outgrowth was resumed at a much slower rate and took a significantly longer time, i.e., 25-30 days to reach ˜80% confluence. The reversible outgrowth from 10 μM U0126 treatment was faster than those that treated with 50 μM LY294002 or 50 μM SR13668 (25 days vs. 30 days to reach ˜80% confluence). These results indicated that such inhibition was reversible and that the progenitor cells in the limbal explant remained viable and could resume proliferation and migration even being treated by these inhibitors for 17 days.

Western blotting analysis was performed to verify that the respective phosphorylation of these kinases was indeed inhibited following the treatment of the aforementioned inhibitors. The results showed that addition of 50 μM of LY294002 or SR13668 abolished phosphorylation of Akt at Thr308 and Ser473. 50 μM SR13668 also abolished while 50 μM LY294002 decreased Thr32 phosphorylation of FKHRL1, a downstream target of Akt. 10 μM U0126 eliminated phosphorylation of Akt at Thr308 and decreased phosphorylation of Akt at Ser473 and FKHRL1 at Thr32. Only 10 μM U0126 abolished phosphorylation of p44/42 MAPK at both Thr202 and Tyr204, while 50 μM LY294002 and 50 μM SR13668 did not change p44/42 MAPK phosphorylation. 10 μM SB203580 and 10 μM JNK inhibitor 1 did increase the phosphorylation of p44/42 MAPK. Interestingly, phosphorylation at Thr180/Tyr182 of p38 MAPK was expressed by cells expanded on plastic, but markedly downregulated in those expanded on intact AM, and abolished with addition of 10 μM SB203580. Likewise, phosphorylation of Thr183/Tyr185 of JUN MAPK was expressed by cells expanded on plastic, but abolished in those expanded on intact AM with or without addition of JNK inhibitor 1. These data collectively further supported that selective activation of Akt and/or p44/42 MAPK without concomitant activation of p38 and JUN MAPKs is uniquely involved in ex vivo expansion of human limbal epithelial progenitor cells on intact AM without 3T3 fibroblast feeder layers.

### Example 9

### Ex Vivo Method for Expansion of Limbal Epithelial Progenitor Cells without Abnormal Epidermal Differentiation by Inhibitors of p38 MAP Kinase

Described below are compositions and methods of use for inhibitors of p38 MAP kinase for improved ex vivo culture of cells. The methods enhance cell outgrowth and differentiation relative to cells grown without inhibitors of p38 MAP kinase.

### Methods

Immediately before use, the AM was thawed, washed three times with sterile PBS, and cut into pieces approximately 2.5×2.5 cm in size. For preparation of denuded AM (dAM), membranes were deprived of their devitalized amniotic epithelial cells by incubation with the appropriate media (0.02% EDTA at 37° C. for 1 h) to loosen the cellular adhesion, followed by gentle scraping with a cell scraper.

The tissue was then rinsed three times with DMEM containing 50 μg/ml gentamicin and 1.25 μg/ml amphotericin B. After careful removal of excessive sclera, conjunctiva, iris, and corneal endothelium, the corneoscleral rim is trimmed to obtain limbal tissue cubes 1.5×2.5 mm size. Afterwards, limbal tissue cubes are placed on inserts with intact AM (iAM) or dAM in SHEM medium made of an equal volume of HEPES-buffered DMED containing bicarbonate and Ham's/F12. The medium is then supplemented with 5% FBS, 0.5% dimethyl sulfoxide, 2ng/ml mouse EFG, 5 μg/ml insulin, 5 μg/ml transferrin, 5 ng/ml selenium, 0.5 μg/ml hydrocortisone, 1 nM cholera toxin, 50 μg/ml gentamicin, and 1.25 μg/ml amphotericin B. In order to keep the explants submerged, 2.5 ml medium was added in each insert and the medium was changed every 2 days.

Human limbal tissue cubes can be placed in the alternative on the center of type I collagen coated inserts and cultured in SHEM medium under submerge and airlift manners, of which the later was also added with p38 inhibitor. In the submerge manner, 4 ml culture medium was added to the culture dish to cover the entire limbal explant. In the airlift manner, 2 ml culture medium was added so as to let the insert bottom sit on the air-liquid interface. That is, the explant was exposed to air, but the remaining explant stroma was submerged in the medium meniscus. In the airlift+p38 inhibitor group, p 38 inhibitor SB 203580 was added to culture medium at a concentration of 10 μM. Cultures were incubated at 37° C. under 5% CO₂ and 95% air, and the medium is changed every 2 days. Rabbit limbal explants are also cultured in the same fashion as the human tissue. Limbal tissue cubes are placed on the center of type I collagen coated inserts and cultured under airlift manner in SHEM medium with or without p38 inhibitor SB203580 for 1 week.

### Results

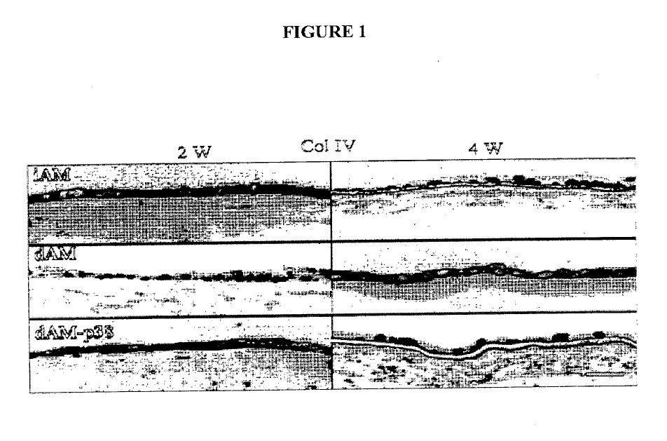

Human corneal limbal explants were cultured on intact amniotic membrane (iAM) or denuded amniotic membrane (dAM) for 2 and 4 weeks. Results showed that outgrowth epithelial cells on iAM started to express type IV collagen intracellurly after 2 weeks of culturing, and deposited the collagen to the epithelial-stromal interface after 4 weeks of culturing (FIG. 1). In contrast, outgrowth epithelial cells on dAM showed negative staining after 2 weeks of culturing, and showed intracellular staining after 4 weeks of culturing. However, when the explant was cultured on dAM in the same culture medium with addition of p38 inhibitor SB203580 for 2 weeks, outgrowth epithelial cells already showed intracellular staining of type IV collagen, and showed strong linear band staining, or deposition at the epithelial-stromal interface, at 4 weeks of culturing. These results indicate that p38 inhibitor can facilitate the basement membrane formation of corneal limbal epithelial cells expanded on dAM.

To study the effect of p38 inhibitor on the outgrowth epithelial sheet, rabbit corneal limbal explant is cultured on the type I collagen coated insert under the airlift manner in SHEM medium for 1 week with or without addition of p38 inhibitor (FIG. 2). The results showed the outgrowth rate was not inhibited by the p38 inhibitor, SB203580, based on the outgrowth surface area.

When explants are cultured in SHEM medium in the airlift manner, the outgrowth epithelial cells exhibited a migratory appearance, which showed a heterogeneous cell shape, a loose outgrowth sheet with space between cells, and an interrupted leading edge. Furthermore, there were stratified cell aggregates in the peripheral area of the outgrowth.

In contrast when explants are cultured in SHEM medium with p38 inhibitor, the outgrowth epithelial cells were uniformly smaller and had a homogeneous cell shape; the outgrowth sheet was very compact and had a very smooth leading edge. H&E staining of the cross-sections showed that the outgrowth sheet without p38 inhibitor was not continuous while the outgrowth sheet was intact and consisted of uniformly two layers when p38 inhibitor was added. This indicated that p38 inhibitor inhibits abnormal epidermal differentiation of limbal epithelial progenitor cells induced by airlifting.

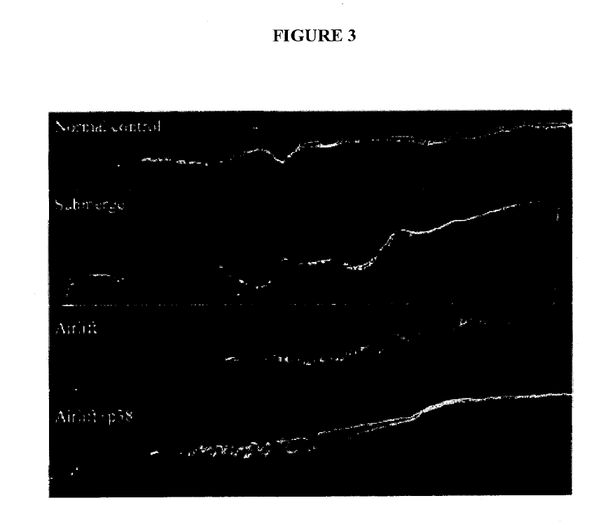

To study the effect of p38 inhibitor on the differentiation of limbal epithelial progenitor cells, human corneal limbal explants were cultured under submerge and airlift manners, of which the later was added with or without p38 inhibitor, on type I collagen coated inserts in SHEM medium for 2 weeks. Afterwards, the explants were embedded in O.C.T., and the cryosections were subjected to immunostaining of K12 and K10. Results show that as reported the normal peripheral cornea epithelium expressed full thickness staining to K12, while the limbal basal cells and some suprabasal cells were K12 negative (FIG. 3). In submerge cultures, epithelial cells migrating to the cut edge of the cornea and on the bare sclera were all K12 positive, while limbal basal epithelial cells still kept K12 negative. In airlift cultures, peripheral corneal epithelial cells still kept positive K12 expression, however, limbal epithelial cells showed island-like groups of cells expressing K12 staining mainly located in the suprabasal epithelium.

Nevertheless, the majority of superficial limbal epithelial cells were K12 negative. Cells on the scleral side were also K12 negative. When explants were cultured in the airlift manner with p38 inhibitor, peripheral corneal epithelial cells also kept K12 expression. Intriguingly, most of the limbal epithelial cells were also K12 positive, including most of the basal cells. However, cells on the scleral side were K12 negative. To further characterize the abnormal differentiation of limbal epithelial progenitor cells in the airlift culture, K10 staining was performed. Results showed that K10 expression was not noted in the normal corneal epithelium, and in the explant cultured in the submerge manner.

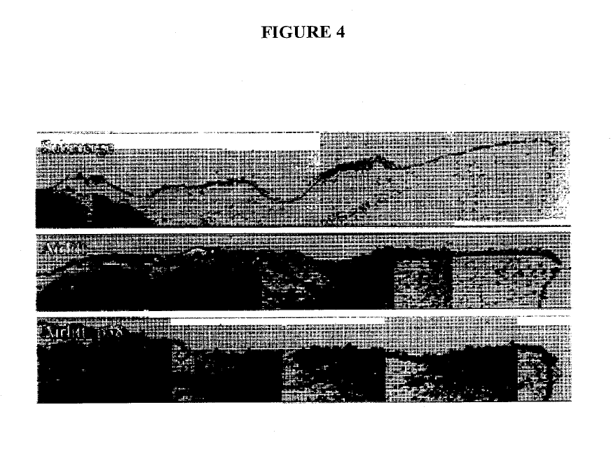

However, when explants were cultured in the airlift manner (without p38 inhibitor) for 2 weeks, groups of cells in the suprabasal and superficial layer expressed K10, and they were only found in the limbal epithelium, but not in the corneal epithelium (FIG. 4). When p38 inhibitor was added into the culture medium, intriguingly, there were no K10 positive cells in both limbal and corneal epithelium. The results indicate that when limbal explants were cultured under the submerge manner, limbal epithelial progenitor cells maintained normal differentiation.

In contrast, when cultured under the airlift manner, some limbal epithelial progenitor cells underwent abnormal epidermal differentiation, which was characterized by the loss of K12 expression, and the gain of epidermal differentiation marker K10 expression. However, when p38 inhibitor was added into the airlift culture, K12 expression was restored in the limbal epithelium and the abnormal epidermal differentiation was blocked.

In one embodiment the p38 MAP kinase inhibitor is a pyridinylimidazole compound. In another embodiment, the p38 MAP kinase inhibitor is a 2,4,5-triarylimidazole. In another embodiment, the p38 MAP kinase inhibitor is chosen from at least one of: SB203580, L 167307, SKF 86002, SB 220025m SB 235699, VX 745, RWJ 68354, SB 202190, SB 239063, and 2-(4-chloro-phenyl)-4-(4-fluoro-phenyl)-5-pyridin-4-yl-1,2-dihydro-pyrazol-3-one. In yet another embodiment described herein, an epithelial sheet outgrowth in a culture medium comprising p38 inhibitor can be used to facilitate engineering of corneal epithelial tissues, both in vitro and in vivo.

### Example 10

### Methods for Expanding Limbal Epithelial Cells and Mesenchymal Cells

### Methods

In order to completely remove the limbal epithelial sheet from the limbus, the limbus has to be digested with Dispase 2 for 18 hours. This notion was verified by the lack of epithelial outgrowth from the remaining stroma after sub-cultured for 2 weeks. In contrast, noted that some limbal basal epithelial cells remained in the stroma when the same Dispase 2 dose was incubated at 37° C. for 1 hour or at 4° C. for 14 hours (not shown). Because it is necessary to incubate in Dispase 2 for such a long period of time, it is important to keep it at a low temperature (4° C.) to reduce metabolic activity, and maintain the tissue in a medium with growth supplements to maintain the viability and in the presence of 100 mM sorbitol to prevent cell swelling by increasing the osmolarity. (See Pfefer B A, “Improved methodology for cell culture of human and monkey retinal pigment epithelium,” Prog Retina Eye Res. 1991;10:251-291, the entire teachings of which are incorporated herein by reference.) By doing so, we confirmed that isolated limbal epithelial sheets indeed retained a high viability of 80.7%. Because human limbal rings used in this study were not fresh and were studied after variable times following death, storing in an Eyebank Storage Medium, and transport to the laboratory, there might have been cell death prior to our digestion. Therefore, we have applied the same digestion protocol to a number of fresh pigmented rabbit limbus, and obtained a mean high viability of 93%.

Because the cleavage plane is within the basement membrane zone, the cell membrane and intercellular junctions remained intact. This not only protected cells from damage, but also maintained the normal cellular phenotype by preserving such intercellular structures as cadherins, integrins, and connexins. Under these conditions, it was noted that the basal epithelium of the isolated limbal epithelium retained pigmentation, did not express keratin 3 and connexin 43, but actively expressed p63. These characteristics are identical to SC features reported in human in vivo limbal epithelium. (See Schermer et. Al, “Differentiation-related expression of a major 64K corneal keratin in vivo and in culture suggests limbal location of corneal epithelial stem cells,” J Cell Biol. 1986;103:49-62. See also Pellegrini, et. al., “p63 identifies keratinocyte stem cells,” Proc Natl Acad Sci USA. 2001;98:3156-3161; and Matic, et. al, “Stem cells of the corneal epithelium lack connexins and metabolite transfer capacity,” Differentiation, 1997;61 :251-260.)

The cleavage plane created by this dispase digestion is different from a brief treatment of 20% ethanol used to prepare an epithelial flap before excimer laser ablation in the procedure of LASEK, of which the cleavage plane is characterized to be located in the lamina lucida and the hemidesmosomes of the basement membrane, and there is ethanol induced cell membrane damage. (See Espana, et. al., “Cleavage of corneal basement membrane components by ethanol exposure like LASEK,” J Cat Refract Surg. 2003; in press.). The final epithelial outgrowth expressed keratin 3 and p63, of which both are regarded as differentiation and proliferation markers of the limbal epithelial SC, respectively. (See Pellegrini G, et. al., “p63 identifies keratinocyte stem cells,” Proc Natl Acad Sci USA. 2001;98:3156-3161.)

With such an intact and viable epithelial sheet isolated from the human limbus, one may begin to study limbal SC with respect to their properties, proliferation and differentiation into the corneal epithelium, and their interaction with the underlying stromal niche. This technique may also facilitate the purification of limbal SC once the surface marker has been identified. Furthermore, it might also be useful to use isolated limbal epithelial sheet to expand limbal epithelial SC ex vivo for therapeutic epithelial transplantation.

### Example 11

### A Reproducible Method of Isolating an Intact Viable Human Limbal Epithelial Sheet; Culturing; and Characterization of Results

### Methods

Human pigmented limbus was incubated at 4° C. for 18 h in SHEM containing 50 mg/ml Dispase 2 and 100 mM sorbitol. A loose limbal epithelial sheet was separated by a spatula. The remaining stroma was digested and subcultured. Viability of isolated cells was assessed. Isolated epithelial sheets and remaining stroma were subjected to immunostaining. Sheets of 1.5 mm length were cultured in SHEM on plastic until confluency and cell extracts were subjected to Western blotting.

### Results

Intact limbal epithelial sheets were consistently isolated. Pigmented palisades of Vogt revealed large superficial squamous cells and small basal cuboidal cells. No epithelial cells grew from the remaining stroma. Mean viability was 80.7±9.1%. The basal epithelium was negative to keratin 3 and connexin 43, but was scatter positive to p63. The epithelial sheet showed negative staining to laminin 5 and collagen VII, but interrupted linear basal staining to collagen IV. The remaining stroma showed negative staining to laminin 5, positive linear staining to collagen IV in the basement membrane, and diffuse staining to collagen VII in the superior stroma subjacent to the basement membrane. Western blotting revealed that cells originated from the limbal sheets expressed keratin 3 and p63.

An intact limbal epithelial sheet can be consistently and reproducibly isolated and contains stem cell characteristics in the basal epithelium by degrading laminin 5 and part of collagen IV, and disassembling collagen VII.

### Example 12

### Enzymatic Isolation of Limbal Epithelial Sheets; Culture; Evaluation

### Methods

Plastic cell culture dishes (60 mm) were from Falcon (Franklin Lakes, N.J., USA). Amphotericin B, Dulbecco's modified Eagle's medium (DMEM), F-12 nutrient mixture, fetal bovine serum (FBS), gentamicin, Hank's balanced salt solutions (HBSS), HEPES-buffer, neomycin, penicillin, streptomycin, phosphate buffered saline (PBS), TRIZOL® and 0.05% trypsin/0.53 mM EDTA were purchased from Gibco-BRL (Grand Island, N.Y., USA). A LIVE/DEAD® viability/cytotoxity kit was from Molecular Probes (Eugene, Oreg., USA). Dispase 2 powder was obtained from Roche (Indianapolis, Ind., USA). Tissue-Tek OCT compound and cryomolds were from Sakura Finetek (Torrance, Calif., USA). Other reagents and chemicals including bovine serum albumin (BSA), cholera-toxin (subunit A), collagenase A, dimethyl sulfoxide, hydrocortisone, insulin-transferrin-sodium selenite (ITS) media supplement, mouse-derived epidermal growth factor (EGF), pre-stained broad band SDS-PAGE standard and sorbitol were purchased from Sigma (St. Louis, Mo., USA). An immunoperoxidase staining kit (Vecstatin®) and DAPI containing mounting media (Vectashield®) were obtained from Vector Laboratories (Burlingame, Calif., USA). We obtained the following monoclonal antibodies: keratin 3 (AE5) (ICN, Aurora, Ohio, USA), integrin □4 (Chemicon, Temeluca, Calif., USA), laminin 5 (Accurate Chemicals, Westbury, N.Y., USA), mouse anti collagen VII antibody, rhodamine conjugated rabbit anti-goat antibody and fluorescein-conjugated goat anti-mouse antibody (Sigma, St. Louis, Mo., USA) and a goat polyclonal antibody against collagen IV (Southern Biotech, Birmingham, Ala., USA).

Enzymatic Isolation of Limbal Epithelial Sheets—Twelve pigmented human corneoscleral rims from donors, younger than 50 years old and less than four days post harvesting, were obtained from the Florida Lions Eye bank within 8 hours after penetrating keratoplasty. After corneal transplantation, they were immediately transferred to SHEM medium, which was made of an equal volume of HEPES-buffered DMEM and Ham's F12 containing bicarbonate, 0.5% dimethyl sulfoxide, 2 ng/ml mouse derived EGF, 5 μg/ml insulin, 5 μg/ml transferrin, 5 ng/ml sodium selenite, 0.5 μg/ml hydrocortisone, 30 ng/ml cholera toxin A subunit, 5% FBS, 50 μg/ml gentamicin, and 1.25 μg/ml amphotericin B. They were then transported at 4° C. within 2 h to the laboratory, where the rims were rubbed off the endothelium and the uveal tissue using a cotton tip and cut by a razor blade into four symmetrical segments, each spanning 3 clock hours starting from 12 O'clock. Each segment was incubated at 4° C. in SHEM containing 50 mg/ml Dispase 2 and 100 mM sorbitol for 18 h. Under a dissecting microscope, an already loose limbal epithelial sheet was separated by inserting and sliding a non-cutting flat stainless steel spatula into a plane between the limbal epithelium and the stroma. This maneuver was videophotographed.

Cell Culture of Remaining Stroma—To determine if there was any epithelial cell left, a total of 16 remaining stromal segments from 8 different donor rims were incubated at 37° C. for 20 min in DMEM containing 1 mg/ml collagenase A. After centrifuge to remove the digestion solution, the remnants were cultured for 2 weeks at 37° C. in a DMEM medium containing 10% FBS, 20 mM HEPES, 50 μg/ml gentamicin and 1.25 μg/ml amphotericin B under 5% carbon dioxide humidified environment. The medium was changed every 2 to 3 days. Five segments that were not exposed to Dispase 2 digestion were subjected to the same collagenase A digestion as described above and used as a positive control.

Viability Evaluation—To determine the viability, 6 isolated limbal epithelial sheets from 6 different donor rims were incubated at 37° C. for 5 min in HBSS containing 0.05% trypsin and 0.53 mM EDTA. After a brief pipetting to achieve a single cell suspension, cells were centrifuged at 800×g for 5 min and re-suspended in PBS containing 2 μM calcein AM and 4 μM ethidium homodimer for 45 min at room temperature before cell counting under a fluorescent microscope. A mean percentage of live cells was calculated by counting both dead (red fluorescence) and live (green fluorescence) cells at ten different locations of a plastic dish. Cultured human corneal epithelial cells expanded from limbal explants that were exposed to methanol for 1 h were used as a positive control as dead cells. (See Tseng S C G, Zhang S-H, “Limbal epithelium is more resistant to 5-fluorouracil toxicity than corneal epithelium,” Cornea 1995;14:394-401, the teachings of which are incorporated herein by reference in their entirety).

Immunofluorescent Staining—After incubating the corneoscleral rims in Dispase 2 as described above, one piece of corneoscleral rim without removing the epithelium was embedded in OCT and snap-frozen in liquid nitrogen for 5 μm frozen sectioning. As a comparison, epithelial sheets and remaining stroma were separately subjected to frozen sectioning. After fixation in cold acetone for 10 min at −20° C., immunofluorecence staining was performed as previously described, (See Grueterich M, Espana E, Tseng S C, “Connexin 43 expression and proliferation of human limbal epithelium on intact and denuded amniotic membrane,” Invest Ophthalmol Vis Sci. 2002;43:63-71, the teachings of which are incorporated herein by reference in their entirety.) using antibodies against the following antigens: keratin 3 (1:100), connexin 43 (1:100), p63 (1:40), integrin β4 (1:100), collagen IV (1:50), collagen VII (1:100) and laminin 5 (1:100). The primary antibody was detected using a fluorescein conjugated secondary antibody except for collagen IV in which a rhodamine conjugated antibody was used. Sections were mounted in anti-fading solution containing DAPI VECTASHIELD® (Vector Laboratories, Burlingame, Calif., USA), and analyzed with a NikonTe-2000u Eclipse epi-fluorescent microscope (Nikon, Tokyo, Japan).

Characterization of Isolated Epithelial Sheet Outgrowth on Plastic—Segments of isolated limbal epithelial sheets (n=7) of 1.5 mm of arc length were cultured until confluency in 60 mm dishes containing SHEM. To determine the expression of keratin 3, which is regarded as a corneal differentiation marker and p63 nuclear protein, that is a presumed corneal SC marker, proteins of confluent cultures were extracted by TRIZOL®, and precipitated by centrifuging at 12000×g in 100% isopropyl alcohol. After washing and centrifuge for three times, the protein pellet was precipitated with a solution of 95% ethanol containing 0.3 M guanidine hydrochloride. A final wash was performed with 100% ethanol and the protein pellet was air dried for 10 min. Pre-stained broad band SDS-PAGE standard and protein samples were dissolved into 1×SDS loading buffer: 50 mM Tris Cl, pH 6.8, 100 mM dithiothreitol, 2% SDS, 1% bromophenol blue and 10% glycerol. Ten μg of total proteins were electrophoresed in a 7.5% gradient polyacrylamide gel. After electrophoretic transfer to a nitrocellulose membrane, the membrane was immersed for 30 min in TTBS, which contained 0.1% (v/v) TWEEN 20™ in 100 mM Tris, 0.9% NaCl, pH 7.5, followed by 1 h blocking with 5% low fat dry milk in TTBS. TWEEN 20™, also known generically as Polysorbate 20, is a surfactant and spreading agent. Membranes were incubated for 1 h at room temperature with primary antibody against p63 (1:250 dilution) and keratin 3 (1:1000 dilution). After washing with TTBS, each membrane was transferred to a 1:200 diluted solution of biotinylated goat anti-mouse antibody in TTBS containing 1% horse serum. After incubating for 30 min, the membrane was incubated with 1:50 diluted VECTASTAIN ELITE® ABC reagent conjugated with peroxidase for 30 min and developed in diaminobenzidine (DAB) (DAKO, Carpintera, Calif., USA) between one and three min.

### Results

Isolation of Epithelial Sheets—Intact limbal epithelial sheets were consistently removed from 48 limbal segments, demonstrating the procedure's simplicity and reproducibility. Microscopic evaluation of the remaining limbal stromal surface revealed the lack of pigmented tissue. Phase contrast microscopic view of the isolated limbal sheets showed large superficial cells on the surface and small basal epithelial cells on the basal surface of the sheet. The isolated limbal epithelial sheet was easy to handle and could be transferred to a culture dish in a medium using a transfer pipette to maintain the sheet's integrity in all cases.

Culturing the Stromal Remnants after Epithelial Sheet Removal—No epithelial outgrowth was seen in any of 16 limbal stromal remnants that were digested by collagenase A and cultured for two weeks. Instead, abundant fibroblasts grew out of these stromal remnants in every remnant. In contrast, all 5 control samples with an intact limbal epithelium showed a characteristic epithelial outgrowth. These findings confirmed that there was no epithelial cell remaining on the stroma after the above isolation.

Cell Viability—The isolated epithelial sheet was then subjected to a brief trypsin/EDTA treatment to render into single cell suspensions. The mean viability rate of six different samples was 80.7±9.1% (ranging from 66.3 to 90.7%). The positive control of methanol-treated cultured human corneal epithelial cells showed a viability of 0% (i.e., 100% of dead cells).

Characterization of Epithelial Phenotype of Isolated Limbal Sheets—Hematoxylin staining of the isolated limbal epithelial sheet showed a stratified and organized epithelium identical to what has been noted in vivo in the human limbus. This stratified epithelium consisted of superficial large squamous cells, intermediate wing cells, and small basal epithelium, which was associated with pigmentation. The superficial surface was smooth, while the basal surface was undulating. Immunostaining of the isolated limbal epithelial sheet showed strong intracytoplasmic staining to the AE-5 antibody, which recognizes keratin 3, in the full thickness stratified epithelium corresponding to the peripheral corneal epithelium, and suprabasal cell layers of the limbal epithelium. This AE-5 staining pattern showing the basal negativity of keratin 3 has been reported as a proof of limbal epithelial SC. (See Schermer A, Galvin S, Sun T-T, “Differentiation-related expression of a major 64K corneal keratin in vivo and in culture suggests limbal location of corneal epithelial stem cells,” J Cell Biol. 1986; 103:49-62.) The intercellular punctuate staining of connexin 43 was found in the suprabasal cells, but absent in the basal cells, a pattern noted previously as well. (See Matic M, et al., “Stem cells of the corneal epithelium lack connexins and metabolite transfer capacity.” Differentiation 1997;61:251-260. See also Wolosin J M, et. al., “Stem cells and differentiation stages in the limbo-corneal epithelium,” Prog Retinal & Eye Res, 2000;19:223-255.) The staining to the transcription factor p63, a reportedly limbal SC marker, was located exclusively in the basal cell layer (See Pellegrini G, et. al., “p63 identifies keratinocyte stem cells,” Proc Natl Acad Sci USA. 2001;98:3156-3161.)

Characterization of the Basement Membrane-Adhesion Complex After Complete Digestion—After complete dispase digestion, we analyzed the basement membrane-adhesion complex before the limbal epithelium was separated. Hematoxylin staining showed that the limbal epithelium was loosely adherent to the underlying stroma as evidenced by the spaces created in between. The staining to collagen IV was positive in the blood vessels and the superficial stroma of the limbus with discontinuous staining in the basement membrane area of the basal surface of the loose limbal epithelial sheet. The staining to collagen VII was linearly positive in the superficial stroma of the corneal portion, but was weak in the superficial stroma of the limbus after digestion. Under a higher magnification, the strong lineal pattern of staining was located in the basement membrane zone of the peripheral cornea. Nevertheless, the staining was diffuse in the superficial stroma of the limbus. Staining to laminin 5 was negative in the basement membrane zone of the entire region, suggesting total digestion of this protein during the 18-hour incubation.

Characterization of the Basement Membrane-Adhesion Complex after Epithelial Sheet Isolation—After digestion, we isolated limbal epithelial sheets and then analyzed the sheet and the remaining stroma separately by immunostaining. The staining to integrin β4 was linearly positive on the basal epithelial cell surface, but was absent on the remaining stroma. The staining to laminin 5 was negative on the entire epithelial sheet and negative on the remaining stroma. The staining to collagen IV was sporadically positive on the basal surface of the isolated sheet, but was strongly positive in a lineal pattern on the superficial surface of the remaining stroma. Staining to collagen VII was negative on the isolated limbal sheet, but was diffusely positive in the superficial stroma of the stromal remnant.

Characterization of Epithelial Outgrowth Derived from Isolated Limbal Epithelial Sheets in Culture—One small segment of isolated limbal epithelial sheet in a size of 1.5 mm of arch length was seeded on the center of each 60 mm plastic dish, and cultured in SHEM. Cells rapidly grew out of the sheet and reached the border of the dish in 17.7±3 days. Epithelial cells continued to grow onto the sidewall of the dish to the level where the medium was. Phase contrast microscopy showed that cells appeared to be small in size and formed a compact monolayer. Western blot analysis of proteins extracted from these cells on confluency showed a positive band of p63 at 60 kDa and a positive band of keratin 3 at 64 kDa.

### Example 13

### A Reproducible Method of Expanding Human Keatocytes on Amniotic Membrane and Characterization of Results

### Methods

Human keratocytes were isolated from central corneal buttons by digestion in 1 mg/ml of collagenase A in DMEM and seeded on plastic or the stromal matrix of human amniotic membrane (AM) in DMEM with different concentrations of FBS. Upon confluency, cells on AM were continuously subcultured for 6 passages on AM or plastic. In parallel, cells cultured on plastic at passages 3 and 11 were seeded back to AM. Cell morphology and intercellular contacts were assessed by phase contrast microscopy and LIVE AND DEATH assay, respectively. Expression of keratocan was determined by RT-PCR and Western blotting.

### Results

Trephined stroma yielded 91,600±26,300 cells (ranging from 67,000 to 128,000 cells per corneal button). Twenty-four hours after seeding, cells appeared dendritic on AM but fibroblastic on plastic even in 10% FBS. Such a difference in morphology correlated with expression of keratocan assessed by RT-PCR and Western blot, which was high and continued at least to passage 6 on AM even in 10% FBS, but was rapidly lost each time when cells on AM were passaged on plastic. Fibroblasts continuously cultured on plastic to passage 3 and 11 did not revert their morphology or synthesize keratocan when re-seeded on plastic in 1% FBS or on AM. Human keratocytes maintain their characteristic morphology and keratocan expression when subcultured on AM stromal matrix even in the presence of high serum concentrations. This method can be used to engineer a new corneal stroma.

### Example 14

### Isolation and Culture of Human Keratocytes on Plastic or AM Membranes; Analyses

### Methods

The tissue culture plastic plates (six-well) and 30 mm culture dishes were from Becton Dickinson (Lincoln Park, N.J.). Culture plate inserts used for fastening AM were from Millipore (Berford, Mass.). Amphotericin B, Dulbecco's modified Eagle's medium (DMEM), fetal bovine serum (FBS), gentamicin, Hank's balanced salt solutions (HBSS), HEPES-buffer, phosphate buffered saline (PBS), 0.05% trypsin/0.53mM EDTA, and TRIZOL® reagent were purchased from Gibco-BRL (Grand Island, N.Y.). 4-15% gradient SDS-polyacrylamide gel and horseradish secondary anti-rabbit antibody were from Biorad (Hercules, Calif.). Collagenase A was obtained from Roche (Indianapolis, Ind.). Aminobenzamidine, EDTA tetrasodium salt, guanidine, hydrochloric acid, isopropanolol, chloroform, endo-β-galactosidase, sodium acetate and urea were from Sigma (St. Louis, Mo.). A LIVE AND DEATH Assay® was obtained from Molecular Probes (Eugene, Oreg.).

Isolation of Human Keratocytes—Human corneas stored in humid chambers for less than 4 days were obtained from the Florida Lions Eye Bank (Miami, Fla.). An 8 mm Barron's trephine was used to remove a central corneal button. After scrapping off the corneal epithelium with a cell scraper and peeling off Descemet's membrane, the remaining corneal stroma was cut into 0.5 mm×0.5 mm pieces. These stromal pieces (˜12 per cornea) were then incubated at 37° C. for 45 min in DMEM containing 1 mg/ml collagenase A in a plastic dish. After incubation, collagenase A was removed by pipetting and the digested stromal pieces were incubated in a second aliquot of collagenase A for another 45 min or until the tissue became “smeared” onto the bottom of the dish. The digested tissue was then centrifuged at 800×g for 5 min and resuspended in 1.5 ml of DMEM containing 20 mM HEPES, 50 μg/ml gentamicin and 1.25 μg/ml amphotericin per cornea. This keratocytes-containing cell suspension was then seeded on plastic dishes or the stromal side of the AM.

Primary Culture of Keratocytes on Plastic or Amniotic Membrane—Human AM preserved according to the method described by Lee and Tseng (See Lee S-H, Tseng S C G, “Amniotic membrane transplantation for persistent epithelial defects with ulceration,” Am J Ophthalmol, 1997;123:303-12.) was kindly provided by Bio-Tissue (Miami, Fla.). After thawing, human AM was incubated in HBSS containing 0.1% EDTA for 30 min at 37° C., and the amniotic epithelium was then denuded using an AMOILS® epithelial scrubber (Innova, Toronto, Ontario, Canada). Epithelially denuded AM with the stromal side facing up was tightened to a small plastic insert—32 mm diameter—using a rubber band in a manner similar to what has been reported. See Grueterich M, Espana E, Tseng S C, “Connexin 43 expression and proliferation of human limbal epithelium on intact and denuded amniotic membrane,” Invest Ophthalmol Vis Sci 2002;43:63-71. The keratocyte cell suspension prepared from one corneal button was seeded on each 32 mm insert or a 35 mm plastic dish. They were cultured in a medium containing DMEM supplemented with 10% FBS, and the medium was changed every 2-3 days. In a separate experiment, cultures grown in DMEM containing 10% FBS for 24 h were switched to DMEM containing 10%, 5%, or 1% FBS and cultured for 10 days.

Subculture of Keratocytes on Plastic and Amniotic Membrane—When the primary culture on AM reached 70-80% confluence, cells were dissociated into single cells by incubation in HBSS containing 0.05% trypsin and 0.53 mM EDTA at 37° C. for 20 min, followed by vigorous pipetting. After centrifuging at 800×g for 5 min, cells were re-suspended in DMEM containing 10% FBS, subdivided into 2 equal parts, with one being seeded onto AM stroma and the other on a plastic dish. They were cultured in DMEM containing 10% FBS. The AM culture was subcultured to either AM or plastic culture in the same manner as described above for a total of 6 passages. In parallel, cells grown on plastic in DMEM containing 10% FBS were continuously subcultured at 1:3 split on plastic. Cells on plastic at passage 3 and 11 were seeded on plastic in DMEM containing 1%, 5%, or 10% FBS or on AM stromal matrix in DMEM containing 10% FBS to see if there was any reversibility in morphology and keratocan expression.

Morphological Analysis Using LIVE-DEATH ASSAY®—At each passage on AM or plastic, cell morphology was documented by phase-contrast microscopy, and in some instances analyzed by the staining with LIVE-DEATH ASSAY® according to a methods described by Poole et al. (See Poole Calif., et al., “Keratocyte networks visualized in the living cornea using vital dyes,” J Cell Sci 1993; 106:685-92.), and the manufacturer. Briefly, after the removal of the culture medium, cells were washed twice with HBSS and incubated for 40 min with 0.5 ml LIVE-DEATH ASSAY® consisting of 2 mM calcein-AM, and 4 mM ethidium homodimer in PBS. After washing with PBS, cells were examined by a NikonTe-2000u Eclipse epi-fluorescent microscope (Nikon, Tokyo, Japan).

Reverse Transcription-Polymerase Chain Reaction (RT-PCR)—Total RNA was extracted by TRIZOL® reagent from two 8 mm central corneal buttons, which had been minced with a blade and sonicated at 6000 rpm using a TISSUE TEAROR™ sonicator (Biospec Products INC, Racine, Wis.) as a positive control. Total RNA was similarly extracted from cells cultured on plastic or AM. Total RNA equivalent to 1×10⁵ cultured cells or one corneal button was subjected to RT-PCR based on a protocol recommended by Promega. The final concentration of RT reaction was 10 mM Tris-HCl (pH 9.0 at 25° C.), 5 mM MgCl₂, 50 mM KCl, 0.1% Triton X-100, 1 mM each DNTP, 1 unit/ml recombinant RNase in ribonucleases inhibitor, 15 units AMV reverse transcriptase, 0.5 mg Oligo(dT)15 primer and total RNA in a total volume of 20 ml. The reaction was kept at 42° C. for 60 min. One tenth sample from RT was used for subsequent PCR with the final concentration of PCR reaction being 10 mM Tris-HCl (pH 8.3 at 25° C.), 50 mM KCl, 1.5 mM Mg(OAc)2, 1.25 units of Taq DNA polymerase in a total volume of 50 ml using primers shown in Table 1. The PCR mixture was first denatured at 94° C. for 5 min then amplified for 30 cycles (94° C., 1 min; 60° C., 1 min; 72° C., 1 min) using PTC-100 Programmable thermal Controller (MJ Research Inc, USA). After amplification, 15 ml of each PCR product and 3 ml of 6× loading buffer were mixed and electrophoresed on a 1.5% agarose gel in 0.5× Tris-boric acid-EDTA (TBE) containing 0.5 mg/ml ethidium bromide. Gels were photographed and scanned.

### Results

Morphological Differences in Primary Cultures—Cell suspension obtained after collagenase digestion yielded 91,600±26,300 cells (ranging from 67000 to 128000 cells per corneal button). Within 24 h after seeding, cells attached to plastic and AM matrix and exhibited a distinctly different morphology. On AM stroma, cells were dendritic or stellate in shape and formed a connecting network when grown in the presence of 1% or 10% FBS for one week. Cells on AM matrix projected their dendritic processes in a 3-dimensional pattern. In contrast, cells on plastic dishes were evenly distributed on a flat surface and adopted a mixture of spindle and stellate shapes when cultured in 1% FBS for one week, but appeared uniformly spindle when cultured in 10% FBS. Cells showed continuous proliferation with increasing concentrations of serum. In 10% FBS, cells on plastic reached confluence in 6 days and cells on AM did so in 14-17 days.

To display better the above difference in cellular morphology of these two culture systems, we used LIVE-DEATH ASSAY® to fully demarcate the entire cytoplasm. Indeed, the majority of cells grown on AM stroma in 10% FBS had a triangular-shaped cell body, and their cytoplasm was stretched into many thin dendritic processes. These processes formed extensive intercellular contacts in a three dimensional pattern. In contrast, cells grown on plastic in 10% FBS maintained spindle shaped cytoplasm with no intercellular contact.

Morphological Differences in Continuous Passages—Cells continued to maintain a dendritic morphology with widespread intercellular contacts when continuously passaged from the primary culture so long as they were grown on AM stromal matrix. Similarly, extensive intercellular contacts were also maintained when illustrated by staining with LIVE-DEATH ASSAY®. In contrast, cells immediately adopted a spindle shape within 24 h when subcultured from the primary AM culture to a plastic dish with a marked loss of intercellular contacts. Such a dramatic change in cell morphology from dendritic to spindle was consistently observed each time when cells on an AM culture were subsequently cultured on a plastic dish for a total of 6 passages tested so far.

When we passaged cells that had continuously been cultured on plastic up to passage 3 to AM stromal matrix, we noted that the fibroblastic morphology remained spindle and did not revert to a dendritic morphology. Even if they were cultured on plastic with 1% FBS, such a spindle shape was not changed. The same result was obtained when we used cells continuously cultured on plastic up to passage 10. Expression of keratocan transcript (1,059 bp) by RT-PCR was found in the normal corneal stroma (K), but was not detected in cells on plastic with 1%, 5%, or 10% FBS or on AM with 10% FBS. The expression of GAPDH (573 bp) serves as a loading control.

Keratocan Expression—Reverse Transcription-Polymerase Chain Reaction (RT-PCR)-Total RNA was extracted from cells seeded on plastic and AM, and RT-PCR was used to determine the expression of keratocan transcript, which was at the size of 1059 base pair (bp). In primary cultures, cells grown on plastic barely expressed keratocan transcript in 1% FBS, but rapidly lost keratocan expression in 5% or 10% FBS. In contrast, cells expressed abundant amounts of keratocan transcript in 1%, 5% and 10% FBS, with the highest noted in 5% FBS.

To determine whether such a difference in keratocan expression correlated with the morphological changes noted above, we continued to subculture primary culture of cells on AM for a total of 6 passages. Total RNA was extracted from AM and plastic cultures for up to passage 5. For each passage, cells cultured on AM were equally divided and subcultured on either AM or plastic. All cells were grown in DMEM containing 10% FBS. Using glyceraldehyde-3-phosphate dehydrogenase (GAPDH) (with a size of 573 bp) as a loading control, we noted that cells sub-cultured on plastic (Prior Art) showed reduced expression of keratocan transcript at passages 1 and 2 but did not express keratocan transcript thereafter up to passage 5. In contrast, cells subcultured on AM expressed abundant amounts of keratocan transcript at passages 1 and 2, and continued to do so up to passage 5. Such a dramatic difference was still maintained at passage 6, the last passage tested so far.

Compared to GAPDH (573 bp) as a loading control, keratocan transcript (1,059 bp) was expressed in all AM cultures but largely lost when subcultured from AM to plastic cultures (Prior Art), especially after passage 3. In contrast, expression of lumican transcript (1,015 bp) and collagen III-a1 transcript (568 bp) was detected in both AM and (Prior art) plastic cultures. As expected, normal corneal stroma (K) expressed keratocan and lumican but not collagen III-a1.

Unlike the aforementioned expression pattern of keratocan, transcripts of lumican (1015 base pair (bp)) and collagen III-a1 (568 bp) were uniformly expressed by cells grown on AM and plastic for up to passage 5 (FIG. 12). As a control, the normal cornea stroma (K) expressed keratocan, lumican but not collagen III-a1. The finding that collagen III-a1 is not expressed by normal corneal stroma, but expressed in wounded cornea has been reported.28

Cells continuously cultured on plastic with 10% FBS up to passage 3 did not express any keratocan transcript when subcultured on plastic even in 1% FBS, or seeded back on AM. The same result was obtained for cells continuously cultured on plastic for up to passage 11.

### Example 15

### In Vivo and In Vitro Characterization of HAEC Phenotype

Described below are compositions and methods of use for generating human amniotic epithelial cell feeder layers. The methods provide for ways of generating a feeder layer that allows for animal cells to be grown on it without causing differentiation of the present state of those cells.

### Methods

Human tissue was handled according to the Declaration of Helsinki. Fresh full-term human placentas were procured from healthy mothers after elective Caesarean delivery by Bio-Tissue, Inc. (Miami, Fla.) under a protocol approved by the institutional review board of the Baptist Hospital in Miami. Corneoscleral tissues from human donor eyes were obtained from the Florida Lions Eye Bank (Miami, Fla.) immediately after the central corneal button had been used for corneal transplantation. Cell culture dishes, plates and centrifuge tubes were purchased from Becton Dickinson (Frankin Lakes, N.J.). Dulbecco's modified Eagle's medium (DMEM) ([Ca²⁺]=1.8 mM), F-12 nutrient mixture (F12) ([Ca²⁺]=0.299 mM), fetal bovine serum (FBS), amphotericin B, gentamicin, Hank's balanced salt solutions (HBSS), defined keratinocyte serum-free medium with supplement (KSFM, [Ca²⁺]=0.07 mM), HEPES-buffer, phosphate buffered saline (PBS) and 0.25% trypsin/1 mM EDTA were purchased from Gibco-BRL (Grand Island, N.Y.). Calcium-free DMEM/F-12 mixture, cholera toxin, dimethyl sulfoxide, hydrocortisone, insulin-transferrin-sodium selenite media supplement and mouse-derived epidermal growth factor (EGF) were obtained from Sigma-Aldrich (St. Louis, Mo.).

For growing the human amniotic epithelial cells, supplemented hormonal epithelial medium (SHEM, [Ca²⁺]=1.05 mM) was made of an equal volume of HEPES-buffered DMEM and F12 containing 0.5% dimethyl sulfoxide, 10 ng/ml EGF, 5 μg/ml insulin, 5 μg/ml transferrin, 5 ng/ml sodium selenite, 0.5 μg/ml hydrocortisone, 10⁻¹⁰ M cholera toxin, 5% FBS, 50 μg/ml gentamicin, and 1.25 μg/ml amphotericin B. Calcium chloride solution at 1 M concentration was from Fluka (Steinheim, Switzerland). Mitomycin C was purchased from Roche Diagnostics GmbH (Mannheim, Germany). Dispase II powder was obtained from Roche (Indianapolis, Ind.). Tissue-Tek OCT compound and cryomolds were from Sakura Finetek (Torrance, Calif.). FITC-conjugated goat anti-mouse and TRITC-conjugated rabbit anti-goat secondary antibodies were purchased from Sigma (St. Louis, Mo.). An ABC kit from Vectastain Elite, Vector Labs (Burlingame, Calif.) and a DAB kit with chromogen from DakoCytomation (DakoCytomation, Carpinteria, Calif.) were used for immunohistochemistry. Primary antibodies used in this study included mouse-anti-human antibodies to CK4 (Sigma, St. Louis, Mo.), CK5/6 (Santa Cruz Biotechnology, Santa Cruz, Calif.), CK8 (ICN Biomedicals, Irvine, Calif.), CK14 (Chemicon, Temecula, Calif.), CK17 (Sigma, St. Louis, Mo.), CK18 (DakoCytomation, Carpinteria, Calif.), CK19 (DakoCytomation, Carpinteria, Calif.), pan-cytokeratin (AE1/AE3, DakoCytomation, Carpinteria, Calif.), Ki67 (DakoCytomation, Carpinteria, Calif.), p63 (DakoCytomation, Carpinteria, Calif.), and ABCG2 (Chemicon, Temecula, Calif.); rabbit-anti-human polyclonal antibodies to vimentin (Abcam, Cambridge, Mass.), connexin 43 (Zymed, San Francisco, Calif.) and Musashi-1 (Abcam, Cambridge, Mass.); goat-anti-human polyclonal antibody to CK12 (Santa Cruz Biotechnology, Santa Cruz, Calif.). For cell proliferation assay, a MTT kit was purchased from Roche (Indianapolis, Ind.).

Human placentas were washed twice in HBSS without calcium or magnesium for 5 min each to remove the blood. An intact layer of the amnion was mechanically peeled off from the chorion, cut into pieces of approximately 5×5 cm², and rinsed in HBSS for another 5 min. The amnion was then incubated in with 10 mg/ml Dispase II in KSFM at 37° C. for 15 min to generate a loose single layer of the amniotic epithelium, which was then separated from the underlying stroma by gentle stripping off with jewelry forceps. Isolated epithelial sheets were further digested with 0.25% Trypsin/1 mM EDTA at 37° C. for 15 min; dissociated cells were collected after centrifugation at 2,000 rpm for 5 min. Cell viability was determined by exclusion of trypan blue dye and counted with a hemocytometer.

### Results

HAECs immediately harvested from the fresh placenta could be cultured in all 9 different media that varied in [Ca²⁺], FBS concentration, and supplements of different growth factors. In serum-free, calcium-free or low calcium media, HAECs became small and round with a high nucleus-to-cytoplasm (N/C) ratio, and were scattered around without formation of cell-cell junction. The proliferation rate, indicated by the number of Ki67-positive cells, was extremely low. Interestingly, some cells gradually lost epithelial markers, i.e., pan-CK over time and turned into Vim-expressing cells. The rest of cells either showed a pan-CK(+)Vim(+) phenotype, or a pan-CK(−)/Vim(−) dedifferentiated phenotype, resembling what was observed in vivo.

In serum-free, high-calcium media HAECs remained small and round, and had a high N/C ratio, but gathered to form a cluster or a small sheet. Cell proliferation was still largely halted. Intriguingly, HAECs also maintained their in vivo phenotype, showing as a mixture of pan-CK(+)/Vim(−) cells and pan-CK(+)/Vim(+) cells.

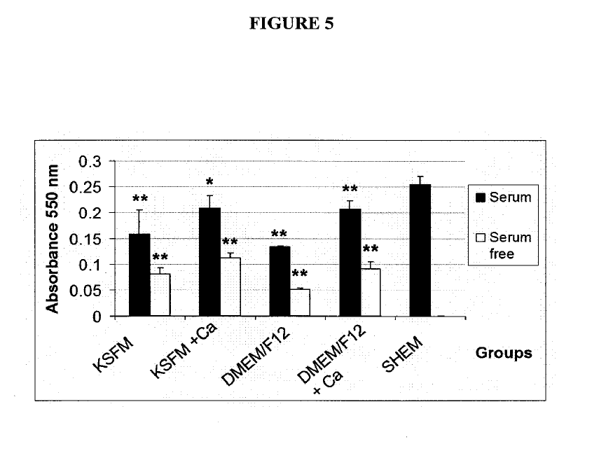

In serum-containing but low-calcium media, HAECs changed to a cobble-stone-like shape with a low N/C ratio, and formed a sheet with obviously discernible intercellular junctions. The proliferation activity was notably increased, as indicated by numerous Ki-67 positive cells. Immunostaining showed that cells expressed both pan-CK and vimentin at the same time with the majority of HAECs exhibiting colocalization of pan-CK and Vim while a minority showing a pan-CK-only phenotype. A fibrillar staining pattern was noted in plethora of cells, while a few cells had a unique perinuclear “vimentin ring”, suggestive of de novo synthesis of vimentin filaments in those cells. Finally, when [Ca²⁺] was increased to 1.08 mM in serum-containing media, the cell morphology and differentiation remained similar to that described above in serum-containing low calcium media. MTT proliferation assay was used to compare the proliferative activity of HAECs in these 9 different media. Similar to K167 staining patterns, addition of FBS significantly promoted cell proliferation when compared to their serum-free counterparts. Increase of [Ca²⁺] in either KSFM or DMEM/F12 increased cell proliferation to a lesser extent. Interestingly, SHEM which contains 5% FBS and other growth supplements yielded the highest proliferation of HAECs (p=0.011 versus condition 4 and p<0.01 versus other 7 conditions, n=5) (FIG. 5).

To confirm if SHEM is indeed the best culture medium to support the growth of HAECs in vitro, we further compared the maximum of cell passage numbers that could be achieved by each culturing medium. In serum-free media, HAECs did not reach confluence and gradually aged by the end of 1-month culturing, and cultures were terminated at Passage 0 (P0). When [Ca²⁺] was increased to 1.05 mM, cell proliferation improved slightly but not enough to be cultured beyond P2. The addition of FBS in low-calcium or calcium-free media or in high-calcium media did not further improve the maximal passage numbers, and cells reached senescence by P3 or P4. Only in SHEM could HAECs actively proliferate, reach confluence in 3 days at 1:3 split, and maintain an epithelial shape for at least P8.

### Example 16

### Preferential Isolation of Limbal Epithelial Stem Cells and Stem-Like Cells by Rapid Adhesion to Collagen

The stem cell population of the corneal epithelium is located in the limbus, the anatomic junction between the cornea and the conjunctiva. In the human limbal epithelium, besides self-renewing stem cells that have a high, if not indefinite, proliferative potential, the other type of proliferating epithelial cells are transit-amplifying cells, which are destined to undergo terminal differentiation after a few rounds of division. Both types of proliferating cells are located in the basal limbal epithelium in contact with the underlying basement membrane, while cells undergoing terminal differentiation migrate to suprabasal cell layers. Such a division of epithelial compartmentalization of proliferative and non-proliferative pools into the basal and suprabasal cell layers is observed in other stratified epithelia including the epidermis. Under normal circumstance, homeostasis maintains these two epithelial pools although the exact regulatory mechanism remains obscure. For example, it remains elusive whether non-proliferating suprabasal epithelial cells affect proliferating progenitor cells. Furthermore, it is not clear whether transient amplifying cells affect the proliferative potential of stem cells.

### Methods

Plastic cell culture dishes (6 well-plates) were purchased from Falcon (Franklin Lakes, N.J.). Rat tail collagen, type I was from BD Biosciences (Bedford, Mass.). Keratinocyte serum-free medium (KSFM), Dulbecco's modified Eagle's medium (DMEM), Ham F-12, fetal bovine serum (FBS), HEPES-buffer, Dulbecco's phosphate-buffered saline (D-PBS), amphotericin B, gentamicin and 0.25% trypsin/EDTA solution were from Invitrogen-GIBCO BRL (Grand Island, N.Y.). Dispase II powder was obtained from Roche (Basel, Switzerland), and mitomycin C (MMC), ITS (bovine insulin, human transferrin, sodium selenite), hydrocortisone, human EGF, cholera toxin, dimethyl sulfoxide (DMSO), heat-denatured bovine serum albumin (BSA), and Hoechst 33342 were from Sigma (St Louis, Mo.). Mouse monoclonal antibodies (mAb) against pan-cytokeratin came from Sigma (St. Louis, Mo.), Ki-67 and p63 from Dako (Fort Collins, Colo.), rabbit anti-Vimentin monoclonal antibody was from abcam (Cambridge, UK), and goat anti-Cytokeratin 12 was purchased from Santa Cruz Biotechnology (Santa Cruz, Calif.). TEXAS RED conjugated donkey anti-mouse and anti-rabbit IgG secondary antibodies were from Jackson ImmunoResearch (West Grovw, Pa.). FITC conjugated goat anti-mouse IgG secondary antibody and FITC conjugated rabbit anti-goat secondary antibody were from Sigma (St Louis, Mo.). Cell proliferation kit I (MTT) was from Roche (Basel, Switzerland).

Isolation of Human Limbal Epithelial Cells—Fresh human corneoscleral tissues from donors younger than 50 years (less than 4 days after death) were obtained from the Florida Lions Eye Bank within 8 hours after corneal transplantation. They were immediately transferred to Optisol-GS and then transported at 4° C. within 2 hours to the laboratory where the tissue was first rinsed with D-PBS containing 50 mg/ml gentamicin and 1.25 mg/ml amphotericin B, the Descemet's membrane and uveal tissue of the corneal rims were removed, and the remaining tissue was cut by a razor blade into symmetric 6 pieces. Each piece was incubated in KSFM containing 10 mg/ml Dispase II at 4° C. for 16 hours or at 37° C. for 2 hours, and the limbal epithelial sheet removed using a spatula and forceps under a dissecting microscope.

Separation of Epithelial Progenitor Cells by Rapid Adhesion—Plastic culture dishes were coated with collagen I (50 μg/ml) (BD Biosciences) for 1 hour at room temperature (RT), and rinsed with D-PBS before use. Isolated limbal epithelial sheets were digested with Trypsin-EDTA at 37° C. for 10 min. After neutralizing with trypsin inhibitor, dissociated single cells were counted using hematocytometer and seeded at 2×10⁴ cells per type I collagen coated well containing KSFM medium. After incubation at 37° C. for 5, 10, 20, and 30 minutes, the unattached cells were removed and transferred to a new collagen-coated dish. Some of these unattached cells, termed slow adherent cells, attach to the collagen-coated dish in 24 hours, at which time the conditioned medium was collected after non-adherent cells were cleared by centrifugation (4° C.×1000 rpm for 5 min). For comparison, control cultures, in which slow adherent cells were not removed, were performed in parallel. The media were changed every 2 to 3 days. After the optimal duration for maximizing the recovery of most undifferentiated progenitor cells was determined, rapid adherent cells, separated from slow adherent cells, were cultured in KSFM on collagen I coated dishes. Upon reaching confluency, these cells were terminated for further characterization.

Subculture of the epithelial cells—The primary cultured cells were subcultured into P1 passage in the following two ways: (a) using the rapid adhesion method (i.e, at every passage, the collagen coated dishes were used and the unattached cells were removed; R+/R+); (b) on collagen coated dishes without removal of the floating cells (R−/R−).

The phenotype and characteristics of the rapid adherent cells (R+/R+) and non-selected cells (R−/R−) were evaluated based on adult stem cell criteria at every passages. The cells were seeded on a 3T3 feeder layer to assess their growth potential. Approximately 4×10³ cells per chamber from each group were seeded into wells of 8-chamber culture slides, incubated at 37° C. overnight, and then fixed for immunofluorescent staining.

Immunofluorescent staining—Limbal epithelial cultures and selected cell populations on 8-chamber slides and 24-well plate were fixed with cold methanol (for cytoplasmic and nuclear protein staining) for 15 min. After blocking with 2% normal goat serum in PBS for 30 min, primary monoclonal antibodies against cytokeratin 12 (1:100), nuclear p63 (1:40), a-SMA (1:100), Ki-67 (1:100), vimentin (1:20), and Pan-cytokeratin (1:100) diluted with 1% normal goat serum in PBS were applied and incubated overnight at 4° C. Appropriate secondary antibodies (1:100) were applied and incubated in a dark chamber for 1 hr at RT, followed by counterstaining with Hoechst 33342 DNA binding dye. After washing with PBS, slides were mounted in antifading solution (Vectashield; VectorbLaboratories) and covered with the cover slip. Slides were examined and photographed with an epifluorescent microscope (TE-2000U Eclipse; Nikon, Japan). The percentages of positively stained cells were calculated by point counting through a Nikon TE-2000U inverted microscope using a 20× objective lens and a 10× subjective lens. A total of 561-1383 nuclei were counted in 6-9 representative fields. The positive index was expressed as the percentages dividing the number of positive staining nuclei by the total number of nuclei.

Colony forming efficiency—MMC treated 3T3 fibroblast feeder layers were used to evaluate proliferative potential of the cell populations selected by adhesion to collagen I. NIH 3T3 cells were cultured in DMEM 10% FBS medium and treated with MMC (4 μg/ml) at 37° C. for 2 hours when they reached to confluence and then trypsinized and plated at a density of 2×10⁴ cells/cm² in 6-well plates. Each cell population was seeded at the density of 500 cells/well onto the sub-confluent 3T3 feeder layer into 6-well culture plates at least in triplicate. The colony forming efficiency (CFE) was calculated at day 12 as a percentage of the number of colonies divided by the number of seeding cells, and the CFE was evaluated by using the staining with crystal violet.

### Results

Enrichment of Progenitor Cells by Optimizing the Method of Rapid Adhesion—To determine whether limbal epithelial progenitor cells can be enriched by optimizing the experimental condition for rapid adhesion, we seeded cells after trypsinization of Dispase-isolated limbal epithelial sheets on collagen type I coated dishes for 5, 10, 20, and 30 min. At the end of each incubation time, non-adherent cells were removed and adherent cells were cultured for 24 h before double immunostaining to K12 keratin, as a marker for suprabasal limbal epithelial cells and corneal transient amplifying cells, and p63, a presumed limbal epithelial progenitor cell marker. As shown in FIG. 8, the percentage of p63-positive nuclei significantly decreased while that of K12 keratin significantly increased according to the time incubation for rapid adhesion. Specifically, the mean percentages of p63-postive nuclei (mean±S.D., n=8) decreased from 55.6±19.8% (5 min) to 53.5±11.8% (10 min), 37.2±7.4% (20 min), and 27.9±5.3% (30 min) (paired t-test; 5 min-10 min: P=0.95, 5 min-20 min: P=0.07, 5 min-30 min: P=0.008). In contrast, the percentage of K12 keratin-positive cells increased from 15.1±8.6% (5 min) to 38.7±11.8% (10 min), 53.9±6.2% (20 min), and 60.4±11.9% (30 min) (paired t-test; 5 min-10 min: P=0.0001, 5 min-20 min: P=6.7×10⁻7, 5 min-30 min: P=0.00001). These results indicate that shorter incubation times, e.g., 5 min, retained more progenitor cells that are positive to p63 but negative to K12 keratin. As a result, limbal epithelial stem cells were preferentially enriched.

Rapid Adherent Cells Indeed Had A higher Proliferative Potential Than Slow Adherent Cells in KSFM—Based on the above results, a 5 min incubation time was used to select rapid adherent cells, while the non-adherent cells were transferred to a new collagen I coated plastic dish. After incubation for 24 hours, the non-adherent cells were removed, and the remaining adherent cells were termed “slow adherent cells”. In KSFM, rapid adherent cells proliferate to form large epithelial clones in 1 week (FIG. 9A). On the contrary, slow adherent cells formed smaller and less organized epithelial clones under the same condition (FIG. 9B). The average clone size for rapid adherent cells was significantly more than that for slow adherent cells, i.e., total number of cells per clone of 121.0±84.6 cells and 17.2±2.8 respectively (FIG. 9C, paired t-test: P=0.007).

At the end of one week of culturing, cells were subjected to immunostaining. The results showed that these two kinds of clones indeed exhibited different levels of expression for Ki-67, p63, K12 keratin, pan-cytokeratins and vimentin. The mean percentage of Ki-67 positive nuclei for clones generated by rapid adherent cells was 86.3±3.2%, which was significantly higher than 46.4±21.4% for slow adherent cells (paired t-test: P=0.007, n=6, FIGS. 9 D, E and H). The mean percentage of p63-positive nuclei for clones generated by rapid adherent cells was 91.0±4.4%, which was significantly higher than 4.6±1.9% for slow adherent cells (P=0.0008, n=8, FIGS. 9 D, F and I). The mean percentage of K12-positive cells for clones generated by rapid adherent cells was 77.5±12.7%, which was significantly higher than 2.4±1.7% for slow adherent cells (P=0.03, n=8, FIG. 9D).

To further evaluate phenotypes of selected rapid adherent cells, immunofluorescent staining with antibodies against vimentin and pan-cytokeratins was performed. As shown in FIGS. 9G and J, there are 3 types of expression pattern in the colonies from rapid adherent cells, (a) positive for both vimentin and pan-cytokeratins; (b) vimentin positive but pan-cytokeratins negative; and (c) only pancytokeratin positive. Most of the vimentin positive cells are small cells, and in particular, the cells that express only vimentin are all small. In other words, the large cells did not express vimentin without pancytokeratins (FIG. 9G). By contrast, the colonies from slow adherent cells were all pan-cytokeratin positive and vimentin negative cells (FIG. 9J).

Rapid adherent cells have significant proliferative potential—To evaluate growth potential, rapid and slow adherent cells were co-cultured with MMC-treated 3T3 fibroblast feeder layer in triplicate at a density of 500 cells/6-well in SHEM medium. The colony forming efficiency (CFE) on day 12 from both rapid and slow adherent cells was 12.4±1.7% and 6.7±0.4%, respectively. CFE of rapid adherent cells in SHEM was significantly higher than that of slow adherent cells (P=0.0005, n=6) (FIG. 10E˜H).

Culturing limbal epithelial cells in low-calcium serum-free KSFM medium also had excellent CFE, suggesting that this system promotes good clonal growth of limbal progenitor cells (FIG. 10A˜D). When the cells were subcultured in KSFM, CFE was maintained with an increase in the passage number. In contrast, the CFE of limbal epithelial cells co-cultured with 3T3 fibroblasts was significantly lower, indicating that the 3T3 system promotes less clonal growth of subcultured limbal progenitor cells.

### Example 17

### Limbal Epithelial Progenitors that Lie Deeper than Dispase Alone can Isolate Require Contact with Limbal Niche Cells for Clonal Growth

Limbal epithelial stem cells (SC), located in the limbal basal layer, are presumably under regulation by the limbal niche. Single epithelial cells obtained from dispase-isolated human limbal epithelial sheets were fractionated by rapid adhesion on collagen I-coated dishes for 12 min before being cultured in KSFM medium. Cells isolated from the remaining limbal stroma by collagenase were briefly treated with or without 0.25% trypsin/EDTA. Their cell phenotypes were characterized by single or double immunostaining to pan-cytokeratins (PCK), vimentin (Vim), p63, and ABCG2, and by clonal growth on plastic in a serum-free KSFM medium or on NIH/3T3 feeder layers in SHEM medium.

Histology and immunostaining to PCK of cross-sections confirmed separation of the limbal epithelial sheet from the underlying stroma by dispase digestion. Dispase-isolated epithelial sheets contained progenitor cells that could readily exhibit clonal growth on 3T3 feeder layers, but could only do so in KSFM when rapidly adherent cells were first selected. Surprisingly, two kinds of clusters made of small PCK+/Vim−/ABCG2+ epithelial aggregates or Vim+/ABCG2+ cells that were in close contact with PCK−/Vim+cells, were found in the collagenase-digested remaining limbal stroma. These clusters generated vivid clonal growth and primarily contained smaller PCK+ epithelial cells with abundant expression of p63 and ABCG2 and Vim+ spindle mesenchymal cells at day 10 in KSFM. In contrast, fewer and larger PCK+ epithelial cells without Vim+ cells were generated by the rapid adherent fraction of isolated epithelial sheets. These clusters also generated better clonal growth on 3T3 feeder layers than cells from epithelial sheets. Once such cell clusters were dissociated by trypsin/EDTA, clonal growth was abolished in KSFM and markedly reduced in 3T3.

Thus, limbal epithelial progenitor cells might lie deeper than dispase alone can isolate; further such “deeper” cells are in close contact with mesenchymal (niche) cells (both expressing ABCG2). Vivid clonal growth is elicited in KSFM without feeder layers when such close contact is maintained, but abolished when it is dissociated.

### Example 18

### Human Amniotic Epithelial Cells as Novel Feeder Layers for Ex Vivo Expansion of Multipotent Limbal Epithelial Progenitor Cells

Intact cryopreserved amniotic membrane with devitalized human amniotic epithelial cells (HAECs) can, but epithelially-denuded cryopreserved amniotic membrane cannot, help expand human limbal epithelial progenitor cells (HLECs) without murine 3T3 fibroblast feeder layers. Thus, HAECs may be used as non-xenogenic feeder layers. HAECs were isolated from a fresh amnion by enzymatic digestion and serially cultured in different media varying in [Ca²⁺], FBS concentration, and supplements of different growth factors. Feeder cells culturing conditions were investigated by immunostaining to pan-cytokeratins(CK)/vimentin(Vim) and MTT assay. HLEC clonal cultures and subcultures in different media or feeder layers on mitomycin C-treated HAEC feeder layers were compared to those on traditional 3T3 fibroblast feeder layers regarding colony-forming efficiencies, differentiation and stem cell-associated markers.

HAECs uniformly expressed pan-CK and heterogeneously expressed Vim. These two subpopulations of Pan-CK(+)/Vim(+) and Pan-CK(+)/Vim(−) cells were maintained in serum-free media with high calcium, but some HAECs turned into pan-CK(−)Vim(−) only in serum-free media with low or no calcium. In contrast, all HAECs became pan-CK(+)/Vim(+) in serum-containing media with an increase in proliferation, and could be subcultured for at least 8 passages in SHEM medium supplemented with EGF and insulin. Mitomycin C-treated HAEC feeder layers were significantly more effective in promoting clonal growth of HLEC progenitor cells than 3T3 feeder layers as judged by a smaller cell size, less K12 keratin expression, lack of connexin 43 expression, and higher percentages of stem cell-associated markers such as p63, Musashi-1 and ABCG2. Clonally expanded HLECs from HAEC feeder layers could further differentiate into neurons and nestin-positive neuronal progenitors when subcultured in serum-free, feeder cell-free KSFM medium or HAEC feeder layers, while those on 3T3 feeder layers might changed into fibroblasts.

These results indicate that HAECs can be used as an xeno-free feeder layer for ex vivo expansion of HLEC progenitor cells. Furthermore, this surrogate niche also fosters the multipotency of the progenitor cells.

It is to be understood that while the disclosed embodiments have been described in conjunction with detailed descriptions thereof, the foregoing descriptions are intended to illustrate and not limit the scope of what has been disclosed, which is defined by the scope of the appended claims. Other aspects, advantages, and modifications are within the scope of the following claims.

What is claimed:

1. An ex vivo method of expanding animal cells comprising expanding animal cells in a culture system comprising a culture vessel, a matrix and a medium, wherein the medium is essentially free of amniotic membrane and non-human mesenchymal feeder cells, under conditions that downregulate TGF-β signaling in the cells to allow the cells to proliferate without undergoing a change in differentiation state.

2. The method of claim 1, wherein the cells are stem cells or stem-cell-like cells.

3. The method of claim 2, wherein the stem cells are selected from the group consisting of: limbal epithelial progenitor cells, umbilical cord epithelial cells, and amniotic membrane epithelial cells.

4. The method of claim 1, wherein the conditions which downregulate TGF-β signaling in the cells comprises culturing the cells in a medium essentially free of serum and comprising less than about 0.10 mM Ca²⁺.

5. The method of claim 1, wherein the medium comprises serum and a Ca²⁺ concentration greater than about 1.0 mM.

6. The method of claim 1, wherein the agent that downregulates TGF-β signaling in the cells prevents translocation of a Smad protein from the cytoplasm of the cell to its nucleus.

7. The method of claim 1, wherein the agent that downregulates TGF-β signaling in the cells is selected from the group consisting of: an extract of amniotic membrane and a purified component of amniotic membrane.

8. The method of claim 1, wherein the agent that downregulates TGF-β signaling in the cells is a purified component of amniotic membrane selected from the group consisting of: TSG-6, pentraxin (PTX3), thrombospondin, hyaluronic acid (HA), HA-ITI, and lumican.

9. A method for facilitating outgrowth of epithelial sheet formation of cells derived from limbal explants and inhibiting abnormal epidermal cell differentiation, the method comprising culturing cells derived from limbal explants on a matrix in a culture medium comprising a p38 MAP kinase inhibitor in an amount effective to facilitate outgrowth of the epithelial sheet and inhibit abnormal epidermal differentiation of the cells relative to cells grown without p38 MAP kinase inhibitor.

10. The method of claim 9, wherein the matrix comprises denuded amniotic membrane.

11. The method of claim 9, wherein the p38 MAP kinase inhibitor is selected from the compounds in Table 1.

12. An ex vivo method for expanding limbal epithelial stem cells comprising contacting the limbus of an eye with an enzymatic solution to isolate the limbal epithelial sheet, forming a composite of the limbal epithelial sheet and a basement membrane side of an amniotic membrane, further culturing the composite for a period of time and under conditions sufficient to enable the epithelial stem cells to expand.

13. The method of claim 12, wherein the enzymatic solution is Dispase II.

14. The method of claim 13, further comprising contacting a population of cells comprising limbal epithelial stem cells with a surface comprising collagen; incubating the population of cells; removing non-adherent cells; and expanding the adherent cells.

15. A method for purifying stem cells or stem-cell-like cells from a population of cells comprising the steps of contacting a population of cells comprising stem cells and stem-cell-like cells with a surface comprising collagen; incubating the population of cells; and removing non-adherent cells.

16. The method of claim 15 wherein the incubating step is less than 1 hour and the collagen is collagen I.

17. A surgical graft comprising limbal epithelial cells expanded according to the method of claim 12.

18. An ex vivo method of expanding mesenchymal cells comprising contacting a stromal side of an amniotic membrane with at least one type of mesenchymal cell thereby forming a composite of the mesenchymal cell and the amniotic membrane, the composite being further cultured in a serum-free medium for a period of time and under conditions sufficient to enable the mesenchymal cells to expand while maintaining their phenotype.

19. A surgical graft comprising mesenchymal cells expanded according to the method of claim 18.

20. The method of claim 18, wherein the mesenchymal cells are keratocytes

21. An in vitro method for expansion of animal cells comprising culturing such animal cells on human amniotic epithelial cells isolated from the amnion layer of a human placenta.

22. The method of claim 21, wherein the human amniotic epithelial cells comprise a feeder layer.

23. The method of claim 22, wherein the human amniotic epithelial cells are MMC-treated human amniotic epithelial cells, and the animal cells are stem cells or stem-cell-like cells.
